(19) United States
(12) Patent Application Publication
(10) Pub. No.: US 2024/0409624 A1
(43) Pub. Date: Dec. 12, 2024

(54) ANTIBODIES

(71) Applicant: UCB BIOPHARMA SRL, BRUSSELS (BE)

(72) Inventors: DALE STARKIE, SLOUGH, BERKSHIRE (GB); DANIEL JOHN LIGHTWOOD, SLOUGH, BERKSHIRE (GB)

(21) Appl. No.: 18/277,594

(22) PCT Filed: Feb. 15, 2022
(86) PCT No.: PCT/EP2022/053694

§ 371 Date: Aug. 17, 2023

(30) Foreign Application Priority Data
Feb. 17, 2021 (GB) 2102227.2

(51) Int. Cl. C07K 16/18; A61P 25/28; G01N 33/68

(52) U.S. Cl. CPC C07K 16/18; A61P 25/28; G01N 33/6896; C07K 2317/33; C07K 2317/565; C07K 2317/82

(57) ABSTRACT

The present invention provides an antibody or antigen-binding fragment thereof that: (a) specifically binds 4R tau protein isoforms in a physiological sample; or (b) specifically binds 3R tau protein isoforms in a physiological sample. The present invention further provides nucleic acids and vectors encoding such an antibody or antigen-binding fragment, as well as host cells comprising such nucleic acids and vectors. The antibodies and fragments may be used in treating and diagnosing tauopathies.

### CROSS-REFERENCE TO RELATED APPLICATION

This application is the U.S. national stage application of International Patent Application No. PCT/EP2022/053694, filed Feb. 15, 2022.

### REFERENCE TO SEQUENCE LISTING

The Sequence Listing for this application is labeled “Seq-List.txt” which was created on Aug. 9, 2023 and is 31,948 bytes. The entire content of the sequence listing is incorporated herein by reference in its entirety.

### FIELD OF INVENTION

The present invention relates to antibodies specific for particular tau proteins, and in particular such antibodies that are specific for 4 R or 3 R tau protein isoforms. The antibodies may be, for instance, used in detection, diagnostics, and therapeutics.

### BACKGROUND OF INVENTION

Microtubule associated protein tau (tau) was discovered in 1975. It was initially characterised as a microtubule binding protein essential for microtubule growth and stabilisation within the axons of neurons. Binding to microtubules is achieved at the C-terminal end of tubulin via the microtubule binding regions (MTBRs) of tau (encoded via exons 9-12). Specifically it has been shown, by peptide competition NMR, that tau interacts with the interface between α- and β-tubulin by residues 224-237, 245-253, 275-284 and 300-317.

The tau gene (MAPT) comprises 16 exons located on chromosome 17q21. Alternative splicing from a single tau pre-mRNA gives rise to six splice variant isoforms of tau within the central nervous system ranging from 342-441 amino acids in length. Tau isoforms are grouped into two categories based on inclusion or exclusion of exon 10 (within the MTBR) giving rise to four MTBRs (4 repeat (4 R) tau) in the presence of exon 10 and three MTBRs (3 repeat (3 R) tau) with the exclusion of exon 10. Within each of these categories there are three N-terminal variants of tau based on the splicing of exons 2 and 3. This gives rise to 0N tau (with neither exon 2 or 3), 1N tau (with exon 2 only) and 2N tau (containing both exons 2 and 3). Tau nomenclature is based upon amino acid numbering from the largest (2N4 R) isoform of tau and as such has four separate functional domains; N-terminal projection (amino acids 1-165); proline rich domain (amino acids 166-242); MTBR (amino acids 243-367); C-terminal domain (amino acids 368-441). The presence or absence of the sequence encoded by exon 10 in the MTBR domain is what denotes 3 R or 4 R tau.

The tau MTBR is a repeating region of the protein which consists of three (3 R tau) or four (4 R tau) repeat regions that are identical in sequence across the isoforms of tau. Each repeat is a different tau exon; repeat 1 (exon 9) (amino acids 242-273); repeat 2 (exon 10) (amino acids 274-304); repeat 3 (exon 11) (amino acids 305-335); repeat 4 (exon 12) (336-367). Whilst there is only 34% complete identical sequence homology across all four repeat regions, the level of homology between amino acids of similar classes (polar, charged, non-charged) or between at least two of the repeat regions is 90%, with repeat region 1 and repeat 4 being the most divergent.

Tau is a largely unstructured protein, but FRET experiments have suggested that the N and C termini are oriented in such a way that they are in close proximity. Further studies using NMR have confirmed that the N and C terminal ends of tau are folded back in a “paperclip” like structure onto the central regions of the protein. It has also been demonstrated that tau has a propensity to form some more complex transient local secondary structure, specifically, β—strands in the MTBR and polyproline helices in the proline rich domain.

Tau is found expressed mainly in neurons and at low levels in oligodendrocytes and astrocytes. Tau was originally identified associated with axonal microtubules and to a lesser extent associated with the plasma membrane, nucleus and mitochondria. In healthy adult neurons the distribution of tau to the axon cross links tubulin and allows interconnection with other cytoskeletal components such as neurofilaments and actin.

These interactions stabilise tubulin assembly into microtubules and regulate their dynamically unstable nature allowing for reorganisation of the cytoskeleton as required for axonal growth. Tau has also been shown to directly regulate the microtubule binding of motor transport proteins dynein and kinesin, which transport cargo in the retrograde and anterograde direction along axons respectively. This regulatory effect is thought to occur via direct competition with the aforementioned motor proteins for access to the microtubules. In tau overexpression systems it is observed a greater effect on kinesin resulting in net retrograde transport and an accumulation of cargo (such as mitochondria) in cell bodies rather than spread out down axons. However, this regulatory effect is not completely understood and may have complementary mechanisms as it has been demonstrated in tau knock-out mice that there is no influence on axonal transport.

Tau protein and its isoforms are involved in a number of diseases typically characterised by the deposition of tau fibrils, with those conditions being collectively known as tauopathies. Although the majority of tauopathies occur sporadically in the population there are many tauopathies that are linked to a MAPT mutation that show the same disease phenotypes as the sporadic counterparts (Ghetti et al., 2015, Neuropathol ApplNeurobiol, 41 (1), 24-46). Tauopathies can be further split into primary and secondary tauopathies. Primary tauopathies are a subgroup of the frontotemporal lobar degeneration (FTLD), which are characterised by neuronal tau inclusions with predominant cell death in the frontal and temporal lobes of the brain. Within these lobes tau inclusions are believed to be the major driving factor in pathology. A well characterised example is progressive supranuclear palsy (PSP). In contrast, secondary tauopathies are diseases where tau pathology is observed in association with other brain pathologies. A well characterised example of a secondary tauopathy is Alzheimer's disease (AD), where disease pathology is characterised by both neuronal tau fibrils and extracellular amyloid plaques, as it was defined by Alois Alzheimer in 1907. It is worth noting that in AD there is still a link between tau load and cognitive decline, indicating the important role tau still plays in this multifactorial disease.

The ratio of tau isoforms appears to play an important role in maintaining tau in a non-pathogenic state. As such, tauopathies can exist as either 3 R tauopathies or 4 R tauopathies characterised by an excess of 3 R or 4 R tau respectively. Pick's disease is a tauopathy predominantly associated with an excess for 3 R-forming filamentous 3 R tau inclusions known as Pick bodies. The major pathology associated with Pick's disease are neuronal and glial loss in the frontal, temporal and parietal lobes of the brain. The much more common form of tauopathy associated with imbalance of tau isoforms are the 4 R tauopathies. An excess of 4 R tau and 4 R tau inclusions has been observed in diseases such as: frontotemporal dementia and parkinsonism's linked to chromosome 17 (FTDP-17); progressive supranuclear palsy (PSP); and corticobasal degeneration (CBD).

Given the importance of tau in a large number of pathological conditions, there is an ongoing need for new diagnostic and therapeutic approaches that target tau.

### SUMMARY OF THE INVENTION

The invention provides an antibody or antigen-binding fragment thereof that:
(a) specifically binds 4 R tau protein isoforms in a physiological sample; or
(b) specifically binds 3 R tau protein isoforms in a physiological sample.

Where the antibody or antigen-binding fragment thereof specifically binds 4 R tau protein, it may:
(a) bind to the amino acid region encoded by exon 10 of the 4 R tau protein when the 4 R tau protein contains a post translational modification at one or more of amino acid positions 279, 280, 281, 285, and 289 of tau 4 R;
(b) bind to a peptide comprising, or consisting of, amino acids 294 to 302 of 4 R tau protein;
(c) bind to a peptide corresponding to a single epitope from the amino acid sequence encoded by exon 10 of tau; or
(d) cross-block or be cross-blocked by any of the antibodies or fragments of (a) to (c).

The antibody or antigen-binding fragment thereof may specifically bind 4 R tau protein isoforms in cell lysates from cells expressing physiological 4 R tau protein isoform(s), preferably in cell lysates from iPSC derived neuronal cells expressing 4 R tau protein isoform(s). The antibody or antigen-binding fragment may detect 4 R tau protein isoforms via immunofluorescence on, or in, cells expressing 4 R tau protein isoform(s).

The antibody or antigen-binding fragment thereof to 4 R tau protein may:
(a) comprise one or more of the light chain CDR1, CDR2, and CDR3 sequences of SEQ ID NOs: 3, 5, and 7 and one or more of the heavy chain CDR1, CDR2, and CDR3 sequences of SEQ ID NOs: 11, 13, and 15, and preferably comprise the light chain CDR1, CDR2, and CDR3 sequences of SEQ ID NOs: 3, 5, and 7 and heavy chain CDR1, CDR2, and CDR3 sequences of SEQ ID NOs: 11, 13, and 15;
(b) comprise the light and heavy chain variable regions of SEQ ID NOs: 1 and 9;
(c) be an intrabody, preferably comprising the sequence of SEQ ID NO: 17 or a sequence with at least 95% sequence identity thereto that is still able to specifically bind tau protein 4 R isoforms; or
(d) cross-block or be cross-blocked by any of the antibodies or fragments of (a) to (c).

Where the antibody or antigen-binding fragment thereof specifically binds 3 R tau protein isoforms, it may:
(a) bind to a peptide, such as a cyclic peptide, that comprises the amino acid sequence encoded by the region bridging exons 9 and 11 of tau; and/or
(b) bind to a peptide, such as a cyclic peptide, consisting of seven amino acids either side of the boundary of the sequence encoded by exons 9 and 11 of tau.

The antibody or antigen-binding fragment thereof to 3 R tau protein may:
(a) comprise one or more of the light chain CDR1, CDR2, and CDR3 sequences of SEQ ID NOs: 20, 22, and 24 and one or more of the heavy chain CDR1, CDR2, and CDR3 sequences of SEQ ID NOs: 28, 30, and 32, and preferably comprise the light chain CDR1, CDR2, and CDR3 sequences of SEQ ID NOs: 20, 22, and 24 and heavy chain CDR1, CDR2, and CDR 3 sequences of SEQ ID NOs: 28, 30, and 32; (b) comprise the light and heavy chain variable regions of SEQ ID NOs: 18 and 26;
(c) be an intrabody, preferably comprising the sequence of SEQ ID NO: 34 or a sequence with at least 95% sequence identity thereto that is able to specifically bind tau protein 3 R isoforms; or
(d) cross-block or be cross-blocked by any of the antibodies or fragments of (a) to (c).

The present invention also provides an antibody or antigen-binding fragment thereof that specifically binds 4 R tau protein, wherein the antibody or antigen-binding fragment thereof binds an epitope of 4 R tau protein comprising amino acids K294, D295, N296, and 1297, optionally where the epitope further comprises K298 and V300 of 4 R tau protein.

The antibody or antigen-binding fragment thereof may be an intrabody or a degrabody.

The invention also provides a nucleic acid or nucleic acids encoding an antibody or antigen-binding fragment of the invention; a vector or vectors comprising said nucleic acids; and a host cell comprising said nucleic acid or nucleic acids or said vector or vectors.

The invention also provides a pharmaceutical composition comprising an antibody or antigen-binding fragment thereof of the invention, a nucleic acid or nucleic acids of the invention, or a vector or vectors of the invention; and a pharmaceutical carrier or excipient.

The invention also provides methods of detecting 4 R or 3 R tau protein isoforms comprising: (a) contacting a test sample with an antibody or antigen-binding fragment thereof the invention; and (b) detecting binding of the antibody or antigen-binding fragment thereof. The invention also provides a method of determining the levels of 4 R and 3 R tau protein isoforms. The methods may be used to diagnose tauopathies, preferably PSP, CBD, or Pick's Disease.

The antibody or antigen-binding fragment thereof may be used to treat a tauopathy, such as a tauopathy comprising an imbalance between 4 R tau protein isoforms and 3 R tau protein isoforms.

### BRIEF DESCRIPTION OF THE FIGURES

FIG. 1: Tau protein sequence exons 9-12 are shown. The TE9/11 peptide sequence is outlined in the solid lined box. Potentially cross-reactive epitopes at the exon 9-10, 10-11 and 11-12 boundaries are highlighted in the dashed box. The amino acid differences between TE9/11 are highlighted by asterisks.

FIG. 2: Tau protein sequence exons 9-12 are shown. The TE10 peptide sequence is outlined in the solid lined box. Potentially cross-reactive epitopes within exons 9, 11 and 12 are highlighted in the dashed boxes. The amino acid differences between TE10 are highlighted via the asterisks. The documented post translational modifications at N279, K280/281 and S285/289 are highlighted in the dotted line boxes to the immediate left of the solid lined boxed region.

FIG. 3: Primary B-cell culture supernatant screening homogenous fluorescence-based assay. Green points represent those that were selected for hit-picking and those in red were not selected.

FIG. 4: TE10 v TE9/11 ELISA data displayed as fold change over background, selected wells for 3 R specific marked in red in the upper left of the graph and for 4 R specific marked in green in the lower right.

FIG. 5: 0N3 R v 0N4 R ELISA data displayed as fold change over background, selected wells for 3 R specific marked in red in the upper left and for 4 R specific marked in green in the lower right FIG. 6: Neat TAP IgG expression products binding ELISA, one representative TAP expression per foci group. Values are expressed as fold change over background binding. 3 R and 4 R specific antibodies selected for cloning are highlighted in red in the upper left and green in the lower right respectively. Black spot represents a well containing an antibody not selected for cloning.

FIG. 7: A ELISA optical density (OD) at 630 nm for the cloned transients of each of the rabbit isoform-specific antibodies. This represents the signal at 10 μg/ml for each of the clones. 4 R-selective antibodies are shown in green whilst the 3 R-selective antibodies are highlighted in red. Clone 2 is not shown as it repeatedly failed to express. Circled in red and green respectively are clone 14 (VR7081) and clone 3 (VR7082) that were selected as the preferred isoform-specific antibodies for further study. B Heavy and light chain CDR3 sequencing of the cloned rabbit antibodies. Highlighted in green and red boxes respectively are the CDR3s of the antibodies selected to be 3 R- and 4 R-specific via ELSA.

FIG. 8: Full titration of VR7081 (A) and VR7082 (B) against all tau isoforms directly coated onto ELISA plates. VR7081 is selective for the tau 3 isoforms and VR7082 for the tau 4 isoforms.

FIG. 9: Flow cytometry assay, VR7081 (A) and VR7082 (B) titrated against intracellular expressed tau isoforms.

FIG. 10: Western blot from single isoform overexpressing cell lysates probed with VR7081 (A) or VR7082 (B).

FIG. 11: Western blot against 2N3 R or 2N4 R tau containing lysates blotted with non-tau reactive rabbit (A) or mouse (A) IgG.

FIG. 12: Western blot from single isoform overexpressing cell lysates probed with VR7081 (A) or VR7082 (B) formatted as Mouse IgGs.

FIG. 13: Immunofluorescent staining of CHOK1 cells expressing 0N3 R or 0N4 R tau co-stained with VR7081(AF647) and polyclonal anti-total tau antibody (AF488).

FIG. 14: Immunofluorescent staining of CHOK1 cells expressing 0N3 R or 0N4 R tau co-stained with VR7082(AF647) and polyclonal anti-total tau antibody (AF488).

FIG. 15: Immunofluorescent staining of iPSC derived neurons with VR7081. Immunofluorescent staining of non-mutant (control) iPSC derived neurons and monoallelic 10+16 MAPT mutant neurons stained with VR7081 (3 R-tau) AF647. In the merged image DAPI is shown in blue whilst tau staining is shown in red. Green arrows denote examples of axonal staining with VR7081 FIG. 16: Immunofluorescent staining of iPSC derived neurons with VR7082 Immunofluorescent staining of non-mutant (control) iPSC derived neurons and monoallelic 10+16 MAPT mutant neurons stained with VR7082 (4 R-tau) AF647. In the merged image DAPI is shown in blue whilst tau staining is shown in red. Green arrows denote examples of axonal staining with VR7082 FIG. 17: Simple Western immunoblots probed with either VR7081 (A) or VR7082 (B).

FIG. 18: Western blot on human brain lysate and recombinant tau ladder probed with VR7081 (A) and VR7082 (B).

FIG. 19: scFv mouse Fc conversion of VR7082 binding profile confirmation. Rabbit IgG (A) and scFv-Ms-Fc (B) format diagrams. VR7082 Rabbit IgG ELISA (C) flow cytometry (E) binding assays, and Western blot (G). VR7082 scFv-Ms-Fc ELISA (D), Flow cytometry (F) binding assays and Western blot (H).

FIG. 20: VR7082 scFv-GFP intrabody testing. A Flow cytometry fluorescence for VR7082-scFv-GFP transfected cells and mock transfected cells. B Western blot from four replicate transfections with VR7082-scFv-GFP. C Western blot of lysate pull down from four replicate VR7082-scFv-GFP transfected cells, pulled down with either TE10 or control peptides. D Western blot band densitometry from VR7082-scFv-GFP pull downs.

FIG. 21: Degradation screen with potential VR7082 degrabody constructs. A Representative Western blot for total tau and GAPDH following co-transfection of 0N4 R tau with degrabody or control intrabody fusions. B Western blot band densitometry of N =3 Western blots.

FIG. 22: Flow cytometry assay of degradation with VR7082-degrabody. A Representative flow cytometry gating and histogram plots demonstrating levels of tau staining for a GFP control, non-degrading intrabody (A-1), a degrabody (A-2) and a degrabody in the presence of MG132 (A-3). B 0N4 R and 0N3 R tau staining, expressed as a percentage of the GFP, non-degrading, intrabody control for all degrabody constructs in the presence and absence of MG132.

FIG. 23: VR7082-XIAP degradation test in iPSC derived neurons. A Gating strategy for GFP based sorting A-1 Identification of live cells via ToPro-3 exclusion. A-2 Identification of cells from debris/cellular vesicles via FSC-A v SSC-A. A-3 GFP positive and GFP negative sort gates. A-4 Overlay plot of sort gating with VR7082-XIAP IRES GFP AAV treated cells (Green) and un-treated cells (Red). B GFP negative sorted cells Peggy Sue Simple Western revealed with polyclonal anti-tau antibody. C GFP positive sorted cells Peggy Sue Simple Western revealed with polyclonal anti-tau antibody.

FIG. 24: VR7082-XIAP degrabody mitochondrial membrane polarisation assay in iPSC derived neurons. A Representative gating strategy for iPSC derived neurone mitochondrial polarisation assay. A-1 identification of nucleated cells via DAPI staining. A-2 identification of intact cells via FSC-A v SSC-A. A-3 Identification of neurons via βIII tubulin staining. A-4 Mitochondrial membrane polarisation assessment. B Representative mitochondrial membrane polarisation histograms derived from the ratio of mitochondrial polarisation/total mitochondrial load. B-1 WT cell iPSC derived neurons. B-2 10+16 monoallelic 10+16 MAPT mutant iPSC derived neurons (10+16 mono). B-3 A12 biallelic MAPT mutant iPSC derived neurons (10+16 biallelic) C Mitochondrial polarisation assay across each of the 3-iPSC derived neuronal lines and AAV treatment groups. ANOVA used to determine statistical significance between groups. D Normalised 3 R tau staining across each of the 3-iPSC derived neuronal lines and AAV treatment groups. ANOVA used to determine statistical significance between groups.

FIG. 25: Comparison of structures of the different tau isoforms. Shows the exons sequences present in different isoforms of Tau protein.

FIG. 26: VR7082 antibody epitope analysis. The graph shows the relative binding of VR7082 antibody to various Tau mutants compared to binding to 0N4 R tau expressed as a percentage. The results demonstrate that the binding of VR7082 to tau is reliant on each of K294; D295; N296; and 1297. When any of those residues are independently mutated to Alanine there is a complete ablation of antibody binding to 0N4 R tau. The results also show that binding is partially reliant on K298 and V300 as binding is decreased by around 50% upon mutation of those positions to alanine. The results show that H299, P301 and G302 are not involved in the binding of VR7082 to tau as when they are independently mutated to alanine there is no difference between the binding levels observed between the alanine mutants and 0N4 R tau. Finally, the results show that VR7082 is capable of binding both P301S and P301L forms of tau protein.

### DETAILED DESCRIPTION OF THE INVENTION

### Tau Proteins

The antibodies and antigen-binding fragments thereof provided by the present invention bind tau proteins. In one particularly preferred embodiment, the tau proteins referred to herein are human tau proteins. The convention of amino acid numbering based on the largest human isoform of tau protein, namely the 2N4 R isoform, is adopted herein. The amino acid sequence of the 2N4 R isoform is provided as SEQ ID NO: 35. As further discussed herein, the antibodies and fragments provided are specific for either 4 R tau protein isoforms or 3 R tau protein isoforms.

### Antibodies Specific for Particular Tau Proteins

The present invention provides antibodies and antigen-binding fragments thereof that are specific for either 4 R tau protein isoforms or are specific for 3 R tau protein isoforms. As discussed further below, typically the antibodies and antigen-binding fragments thereof that are specific for the 4 R tau protein isoforms bind to a region of 4 R tau protein isoforms that comprises at least part of the region encoded by exon 10 of the tau gene (the MAPT gene) as that region is unique to 4 R tau protein isoforms. Conversely, typically antibodies or antigen-binding fragments of the present invention that are specific for 3 R tau protein isoforms recognise a portion of the protein that includes that bridging sequence encoded by the junction of exons 9 and 11 of the tau gene.

In one embodiment, a particular advantage of the present invention is that the antibody or antigen-binding fragment thereof binds the 4 R or 3 R tau protein isoforms in a physiological state, for example where the protein is not in denatured form. For instance, an antibody or an antigen-binding fragment thereof of the present invention may specifically bind 4 R or 3 R tau protein isoforms in intact cells, for example as determined by immunofluorescence. In one embodiment, it is able to detect the protein in flow cytometry analysis of cells. In another embodiment, it may be used to detect the protein in a tissue sample, for example by immunofluorescence. In another, it may be able to detect the protein via immunohistochemistry. In a preferred embodiment, it may be possible to detect the tau protein via Western blot and in particular by Peggy Sue Simple Western.

In one embodiment, “specifically binds” as used herein means that the antibody or antigen-binding fragment thereof binds at least 10 times more strongly to whichever of 4 R and 3 R tau isoforms it is specific for. For example, it may bind more than 50, 100, 200, or 500 times more strongly to 4 R tau than 3 R tau protein isoforms. In another embodiment, it may bind more than 50, 100, 200, or 500 times more strongly to 3 R tau than 4 R tau protein isoforms. In one embodiment, the binding is at least 1000 times stronger for whichever of 4 R and 3 R tau protein isoforms it is specific for. In one embodiment, the K_{D} value for binding is at least 10, 50, 100, 500 or 1000 times less for whichever of the 3 R or 4 R tau protein it is specific for. In another embodiment, the binding may be 5,000, 10,000, or 50,000 times stronger for the form of tau protein for which it is specific. In one embodiment, the antibody or antigen-binding fragment thereof of the present invention does not bind the other form of tau protein at all or does not significantly do so. In another embodiment, the level of binding to non-tau proteins is at least as low as the level of binding to the form of tau for which the antibody is not specific. In one embodiment, the antibody or antigen-binding fragment thereof does not bind non-tau proteins or does not significantly do so. Levels of binding may be measured by techniques well known in the art such as surface plasmon resonance or any of the other relevant techniques disclosed herein. In one embodiment, the level of specificities set out above are in relation to specificity for 4 R tau protein isoforms over 3 R tau protein isoforms. In another embodiment, the level of specificities set out above are in relation to specificity for 3 R tau protein isoforms over 4 R tau protein isoforms.

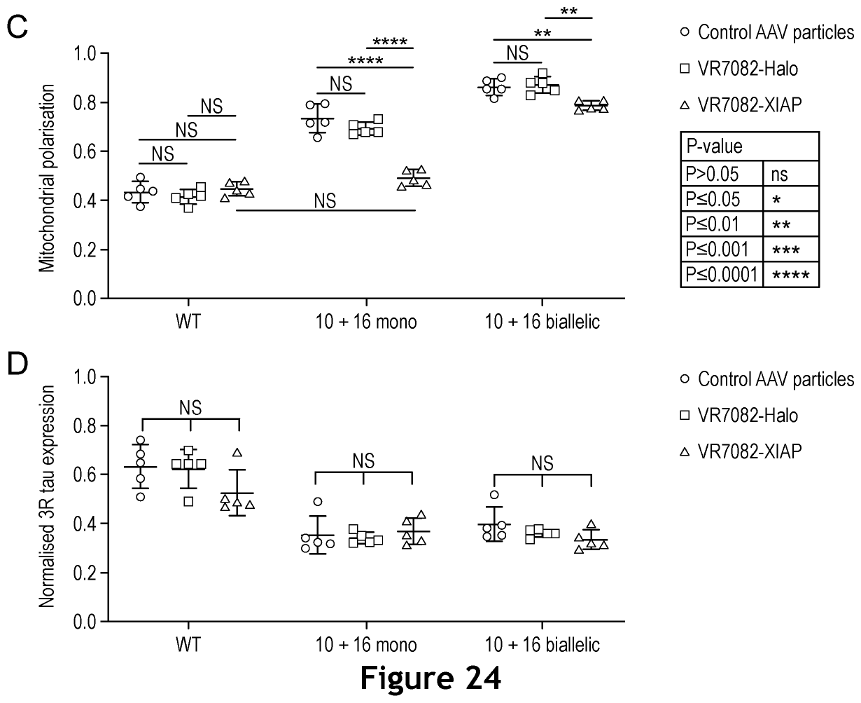

As shown in FIG. 25, 4 R tau protein isoforms represent a group that all have the amino acid sequence encoded by exon 10 of the tau gene, whereas 3 R tau protein isoforms represent a group of isoforms that have the common feature of all lacking the amino acid sequence encoded by exon 10. Those features denote how tau proteins are grouped into 4 R and 3 R tau protein isoforms, but within each of those designations there are isoforms that differ depending on whether or not the amino acid sequence encoded by exons 2 and 3 are present or absent. So, for instance, 4 R tau protein isoforms are: 2N4 R which has the sequence encoded by exons 2 and 3; 1N4 R which has the sequence encoded by exon 2, but not that encoded by exon 3; and 0N4 R which has neither the sequence encoded by exon 2 nor that encoded by exon 3. All three of 2N4 R, 1N4 R, and 0N4 R though include the sequence encoded by exon 10 and so are designated 4 R tau proteins. Hence, where reference is made herein to an antibody or antigen-binding fragment thereof that specifically binds 4 R tau protein isoforms it typically means that the antibody or fragment binds all three of 2N4 R, 1N4 R, and 0N4 R, but not any of the 3 R tau protein isoforms 2N3 R, 1N3 R and 0N3 R. In embodiments where the antibody or antigen-binding fragment is one specifically binding 3 R tau protein isoforms, the converse will be the case and so the antibody will bind 2N3 R, 1N3 R and 0N3 R, but not 2N4 R, 1N4 R, and 0N4 R.

In one embodiment, the affinity of the antibody or antigen-binding fragment thereof for whichever of 4 R or 3 R tau protein isoforms that is specific is about 100 nM or stronger, such as about 50 nM, 20 nM, 10 nM, 1 nM, 500 pM, 250 pM, 200 pM, 100 pM or stronger. In one embodiment, the binding affinity is 50 pM or stronger. In one embodiment, the affinity of the antibody for whichever of 4 R or 3 R tau protein it is specific may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 pM, less than 1 pM, or less than 0.1 pM. In some embodiments, the Kd is from about 0.1 pM to about 1 μM.

Tau proteins include repeats with high levels of sequence identity in the MTBR region. In the case of 4 R, four such repeats are present and, in the case of 3 R, three such repeats are present. In one embodiment, an antibody or antigen-binding fragment thereof of the present invention binds only the repeat containing its epitope and not the other repeats. For example, in one embodiment, an antibody or antigen-binding fragment of the present invention binds to a repeat comprising the junction between exons 9 and 11, but does not bind to the other repeats and so is specific for 3 R tau. In another embodiment, an antibody or antibody fragment of the present invention binds to a sequence within the repeat present in the sequence encoded by exon 10 of the tau gene, but not to the sequence of the other three repeats.

### Tau 4 R Specific Antibodies

In one particularly preferred embodiment, an antibody or antigen-binding fragment thereof of the present invention is specific for tau 4 R protein isoforms. In one embodiment, an antibody or antigen-binding fragment thereof specifically binds tau 4 R protein isoforms and not 3 R tau protein isoforms. For instance, it may bind all isoforms of tau 4 R protein for a given species or subject, but none of the isoforms of tau 3 R protein for that species or subject. Hence, in one embodiment it may bind all of the 2N4 R, 1N4 R and 0N4 R isoforms of tau 4 R protein, but not bind any of the 2N3 R, 1N3 R and 0N3 R isoforms of tau3 R protein. In one embodiment, an antibody or antigen-binding fragment thereof specifically binds tau 4 R protein isoforms from human, rat, and mouse, but does not bind 3 R tau protein isoforms from those species.

The common difference between 4 R tau protein isoforms and those of 3 R tau protein isoforms is that the latter lack the amino acid sequence encoded by exon 10 of the tau gene. In one embodiment, an antibody or antigen-binding fragment thereof that is specific for 4 R tau protein isoforms therefore binds to a region that is unique to 4 R tau protein isoforms and hence to that encoded by exon 10. In one preferred embodiment, the antibody or antigen-binding fragment thereof specific for 4 R tau protein isoforms binds to an epitope that is wholly or partially in the sequence encoded by exon 10 of the tau gene. In one preferred embodiment, an antibody or antigen-binding fragment of the present invention binds to a region within the amino acid sequence encoded by exon 10 of the tau gene and in particular does not bind 3 R tau protein isoforms.

In one embodiment, an antibody or antigen-binding fragment thereof of the present invention which is specific for 4 R tau protein isoforms is able to bind a peptide that comprises at least part of the amino acid sequence encoded by exon 10 of the tau gene. In one embodiment, the peptide sequence is entirely within the region encoded by exon 10 of the tau gene. In one embodiment, the peptide is less than 15 amino acids in length. In one embodiment, it is less than 14, 13, 12, 11, or 10 amino acids in length. In one embodiment, the antibody or antigen-binding fragment thereof binds a peptide sequence which is nine amino acids in length. In one preferred embodiment, it is able to bind a peptide which consists of amino acids 294 to 302 of the 4 R Tau protein isoforms. In one embodiment, the antibody or antigen-binding fragment thereof is able to bind such a peptide when the peptide is in linear form. In one embodiment, an antibody or antigen-binding fragment thereof of the present invention is able to bind a peptide corresponding to an amino acid sequence encoded by exon 10 of the tau gene which is of a length that only has one epitope present. In one embodiment, an antibody or antigen-binding fragment thereof is able to bind such peptides when conjugated to a carrier, for example KLH, ovalbumin, or BSA, particularly where the peptide is in linear form.

In one preferred embodiment, an antibody or antigen-binding fragment thereof is able to specifically bind 4 R tau protein isoforms irrespective of whether or not the protein has any post translational modifications at one or more of amino acid positions 279, 280, 281, 285, and 289. In one embodiment, it is able to specifically bind tau 4 R protein isoforms irrespective of whether or not the protein is modified at any, or all, of amino acid positions 279, 280, 281, 285 and 289. Frequent post translational modifications for these positions include glycosylation, phosphorylation, and acetylation. The most frequent posttranslational modification at position 279 is glycosylation, the most frequent post translational modification at positions 281 and 289 is acetylation, and the most frequent post translational modification at positions 285 and 289 is phosphorylation. In one embodiment, an antibody or an antigen-binding fragment thereof of the present invention is able bind tau 4 R protein isoforms irrespective of whether there is any glycosylation, acetylation, or phosphorylation at those positions. In one embodiment, an antibody or an antigen-binding fragment thereof of the present invention is able bind tau 4 R protein isoforms irrespective of whether or not the protein is glycosylated at position 279, acetylated at position 281, acetylated at position 289, phosphorylated at position 285, and/or phosphorylated at position 289. In other words, the antibody binds tau 4 R protein both when glycosylated, acetylated, and/or phosphorylated at these positions and when not glycosylated, acetylated, and/or phosphorylated at these positions.

In one embodiment, an antibody or antigen-binding fragment specific for the 4 R tau isoforms binds to repeat 2 within the 4 R tau protein sequence, but not to the other three repeats present in the 4 R tau protein isoforms. Each repeat corresponds to a different tau exon; repeat 1 (exon 9) (amino acids 242-273); repeat 2 (exon 10) (amino acids 274-304); repeat 3 (exon 11) (amino acids 305-335); repeat 4 (exon 12) (336-367). Hence, in one embodiment, an antibody may bind to the sequence encoded by exon 10, but not by the sequences encoded by exons 9, 11, and 12 of the 4 R tau protein isoforms.

In one embodiment, the antibody or antigen-binding fragment thereof specific for 4 R tau proteins:
(a) binds to the amino acid region encoded by exon 10 of tau when the tau 4 R protein contains a post translational modification at one or more of amino acid positions 279, 280, 281, 285, and 289 of tau 4 R;
(b) binds to a peptide comprising, or consisting of, amino acids 294 to 302 of tau 4 R protein; and/or
(c) binds to a peptide corresponding to a single epitope from the amino acid sequence encoded by exon 10 of tau; or
(d) cross-blocks or is cross-blocked by any of the antibodies or fragments of (a) to (c).

In one embodiment, an antibody or antigen-binding fragment of the present invention:
(a) comprises one or more of the light chain CDR1, CDR2, and CDR3 sequences of SEQ ID NOs: 3, 5, and 7 and one or more of the heavy chain CDR1, CDR2, and CDR 3 sequences of SEQ ID NOs: 11, 13, and 15, preferably comprising the light chain CDR1, CDR2, and CDR3 sequences of SEQ ID NOs: 3, 5, and 7 and heavy chain CDR1, CDR2, and CDR 3 sequences of SEQ ID NOs: 11, 13, and 15; or
(b) comprises the light and heavy chain variable regions of SEQ ID NOs: 1 and 9;
(c) is an intrabody, preferably comprising the sequence of SEQ ID NO: 17 or a sequence with at least 95% sequence identity thereto that is still able to specifically bind tau protein 4 R isoforms; or
(d) cross-blocks or is cross-blocked by any of the antibodies or fragments of (a) to (c).

In one embodiment, an antibody or antigen-binding fragment of the present invention comprises a light chain CDR1, CDR2, and CDR3 corresponding to the sequences of SEQ ID NOs: 3, 5, and 7, or CDRs where each CDR has no more than two amino acid sequence changes compared to the sequence of SEQ ID NOs: 3, 5, and 7 and the antibody is still able to specifically bind 4 R tau protein isoforms. In one embodiment, the sequence changes are conservative amino acid sequence changes. In another embodiment, an antibody or antigen-binding fragment of the present invention comprises heavy chain CDR1, CDR2, and CDR3 sequences corresponding to the sequences of SEQ ID NOs: 11, 13, and 15, or CDRs where each CDR has no more than two amino acid sequence changes compared to the sequence of SEQ ID NOs: 11, 13, and 15 and the antibody is still able to specifically bind 4 R tau protein isoforms. In one embodiment, the antibody or antigen-binding fragment thereof comprises both such light and heavy chain CDRs and, in a preferred embodiment, comprises the light chain CDR1, CDR2, and CDR3 sequences of SEQ ID NOs: 3, 5, and 7 and the heavy chain CDR1, CDR2, and CDR3 sequences of SEQ ID NOs: 11, 13, and 15. In one embodiment, the light chain of the antibody may comprise one or more of the framework regions of the FW1, FW2, FW3, and FW4 of SEQ ID NOs: 2, 4, 6, and 8. In one embodiment it may comprise all four of the FW1, FW2, FW3, and FW4 of SEQ ID NOs: 2, 4, 6, and 8. In another embodiment, the heavy chain of the antibody may comprise one or more of the framework regions of the FW1, FW2, FW3, and FW4 of SEQ ID NOs: 10, 12, 14, and 16. In one embodiment, it may comprise all four of those framework regions. In another embodiment, it may comprise both such heavy and light chain framework regions. In one embodiment, an antibody or antigen-binding fragment thereof comprising the above sequences is humanized, for example so that all of the sequences of the antibody apart from the CDR sequences are human sequences, with the antibody or antigen-binding fragment thereof still specifically binds 4 R tau protein isoforms. In another embodiment, all of the sequences of the antibody may be human apart from the variable regions.

In one embodiment, an antibody or antigen-binding fragment thereof comprises a light chain variable region of SEQ ID NO: 1 or a light chain variable region with at least 90% sequence identity thereto, where the antibody or antigen-binding fragment is still able to specifically bind 4 R tau protein isoforms. In one embodiment, the light chain variable region has a least 95%, 98% or 99% sequence identity or has 10, 9, 8, 7, 6, 5, 4, 3, 2, or 1 amino acid sequence changes compared to SEQ ID NO: 1, whilst still being able to specifically bind 4 R tau protein isoforms. In one embodiment, an antibody or antigen-binding fragment thereof comprises a heavy chain variable region of SEQ ID NO: 9 or a heavy chain variable region with at least 90% sequence identity thereto, where the antibody or antigen-binding fragment is still able to specifically bind 4 R tau protein isoforms. In one embodiment, the light chain variable region has a least 95%, 98% or 99% sequence identity or has 10, 9, 8, 7, 6, 5, 4, 3, 2, or 1 amino acid sequence changes compared to SEQ ID NO: 9 and still specifically binds 4 R tau isoforms. In one embodiment the sequence changes compared to the heavy and light chain sequences are solely in the framework regions of the variable regions. In one embodiment, an antibody or antigen-binding fragment thereof comprises both a light chain and heavy chain variable region as set out above. In one embodiment, an antibody or antigen-binding fragment of the present invention comprises any of the above light and/or heavy chain variable region sequences, but where they have been humanised so that all of the sequences apart from the CDR sequences are human.

As discussed further below, an antibody or antigen-binding fragment of the present invention may be provided in a variety of antibody and antibody fragment formats, and those which are specific for 4 R tau protein isoforms may be provided in any of the antibody formats set out herein. They may also have any of the levels of sequence variation set out herein and any of the properties set out herein.

### Tau 3 R Specific Antibodies

In a further preferred embodiment, an antibody or antigen-binding fragment thereof of the present invention is specific for 3 R tau protein isoforms. In one embodiment an antibody or antigen-binding fragment thereof specifically binds 3 R tau protein isoforms and not 4 R tau protein isoforms. For instance, it may bind all isoforms of 3 R tau protein for a given species or subject, but none of the isoforms of 4 R tau protein for that species or subject. Hence, in one embodiment it may bind all of the 2N3 R, 1N3 R and 0N3 R isoforms of 3 R tau protein, but not bind any of the 2N4 R, 1N4 R and 0N4 R isoforms of 4 R tau protein.

The common difference between 3 R tau proteins and those of 4 R tau is that the former lack the amino acid sequence encoded by exon 10 of the tau gene. In one embodiment, an antibody or antigen-binding fragment thereof that is specific for 3 R tau protein isoforms therefore binds to a region that is unique to 3 R tau protein isoforms and in particular to a region that includes the amino acid sequence corresponding to the junction between exons 9 and 11 of the tau gene. In one preferred embodiment, an antibody or antigen-binding fragment of the present invention binds to an epitope that comprises said junction between exons 9 and 11.

In one embodiment, an antibody or antigen-binding fragment thereof of the present invention which is specific for 3 R tau protein isoforms is able to bind a peptide that comprises the sequence encoded by the junction between exons 9 and 11. In one embodiment, the peptide is less than 15 amino acids in length. In one embodiment, it is 14 amino acids in length. In one preferred embodiment, it is able to bind a peptide which consists of 14 amino acids, with seven amino acids from each side of the junction between exons 9 and 11. In one embodiment, an antibody or antigen-binding fragment thereof of the present invention which is specific for 3 R tau protein isoforms is able to bind a protein comprising amino acids 268 to 274 and 306 to 312 of SEQ ID NO: 35, but not amino acids 275 to 305 of SEQ ID NO: 35. In one embodiment, an antibody or antigen-binding fragment thereof of the present invention which is specific for 3 R tau protein isoforms is able to bind a peptide that comprises the amino acid sequence of SEQ ID NO: 36. In one embodiment, the antibody or antigen-binding fragment thereof is able to bind such a peptide when the peptide is in cyclic form and in particular when conjugated to a carrier, such as KLH, ovalbumin, or BSA.

In one embodiment, the antibody or antigen-binding fragment thereof specific for 3 R tau protein isoforms:
(a) comprises one or more of the light chain CDR1, CDR2, and CDR3 sequences of SEQ ID NOs: 20, 22, and 24 and one or more of the heavy chain CDR1, CDR2, and CDR 3 sequences of SEQ ID NOs: 28, 30, and 32, and preferably comprises the light chain CDR1, CDR2, and CDR3 sequences of SEQ ID NOs: 20, 22, and 24 and heavy chain CDR1, CDR2, and CDR 3 sequences of SEQ ID NOs: 28, 30, and 32; and/or
(b) comprises the light and heavy chain variable regions of SEQ ID NOs: 18 and 26;
(c) is an intrabody, preferably comprising the sequence of SEQ ID NO: 34 or a sequence with at least 95% sequence identity thereto that is still able to specifically bind tau protein 3 R isoforms;
(d) cross-blocks or is cross-blocked by any of the antibodies or fragments of (a) to (c).

In one embodiment, an antibody of antigen-binding fragment of the present invention comprises a light chain CDR1, CDR2, and CDR3 corresponding to the sequences of SEQ ID NOs: 20, 22, and 24, or CDRs where each CDR has no more than two amino acid sequence changes compared to the sequence of SEQ ID NOs: 20, 22, and 24 and the antibody is still able to specifically bind 3 R tau protein isoforms. In one embodiment, the sequence changes are conservative amino acid sequence changes. In another embodiment, an antibody or antigen-binding fragment of the present invention comprises a heavy chain CDR1, CDR2, and CDR3 corresponding to the sequences of SEQ ID NOs: 28, 30, and 32, or CDRs where each CDR has no more than two amino acid sequence changes compared to the sequence of SEQ ID NOs: 28, 30, and 32 and the antibody is still able to specifically bind 3 R tau protein isoforms. In one embodiment, the antibody or antigen-binding fragment thereof comprises both such light and heavy chain CDRs and, in a preferred embodiment, comprises the light chain CDR1, CDR2, and CDR3 sequences of SEQ ID NOs: 20, 22, and 24 and the heavy chain CDR1, CDR2, and CDR3 sequences of SEQ ID NOs: 28, 30, and 32. In one embodiment, the light chain of the antibody may comprise one or more of the framework regions of the FW1, FW2, FW3, and FW4 of SEQ ID NOs: 19, 21, 23, and 25. In one embodiment it may comprise all four of the FW1, FW2, FW3, and FW4 of SEQ ID NOs: 19, 21, 23, and 25. In another embodiment, the heavy chain of the antibody may comprise one or more of the framework regions of the FW1, FW2, FW3, and FW4 of SEQ ID NOs: 27, 29, 31, and 33. In one embodiment, it may comprise all four of those framework regions. In another embodiment, it may comprise both such heavy and light chain variable regions. In one embodiment, an antibody or antigen-binding fragment thereof comprising the above sequences is humanized, for example so that all of the sequences of the antibody apart from the CDR sequences are human sequences, with the antibody or antigen-binding fragment thereof still being able to specifically bind 3 R tau protein isoforms.

In one embodiment, an antibody or antigen-binding fragment thereof comprises a light chain variable region of SEQ ID NO: 18 or a light chain variable region with at least 90% sequence identity to SEQ ID NO: 18, where the antibody or antigen-binding fragment is still able to specifically bind 3 R tau protein isoforms. In one embodiment, the light chain variable region has a least 95%, 98% or 99% sequence identity or has 10, 9, 8, 7, 6, 5, 4, 3, 2, or 1 amino acid sequence changes compared to SEQ ID NO: 18, whilst still be able to specifically bind 3 R tau protein isoforms. In one embodiment, an antibody or antigen-binding fragment thereof comprises a heavy chain variable region of SEQ ID NO: 26 or a heavy chain variable region with at least 90% sequence identity thereto, where the antibody or antigen-binding fragment is still able to specifically bind 3 R tau protein isoforms. In one embodiment, the light chain variable region has a least 95%, 98% or 99% sequence identity or has 10, 9, 8, 7, 6, 5, 4, 3, 2, or 1 amino acid sequence changes compared to SEQ ID NO: 18 or 26 and is still able to specifically bind 3 R tau protein isoforms. In one embodiment the sequence changes compared to the heavy and light chain sequences are solely in the framework regions of the variable regions. In one embodiment, an antibody or antigen-binding fragment thereof comprises both a light chain and heavy chain variable region as set out above. In one embodiment, an antibody of antigen-binding fragment of the present invention comprises any of the above light and/or heavy chain variable region sequences, but where they have been humanised so that all of the sequences apart from the CDR sequences are human.

As discussed further below, an antibody or antigen-binding fragment of the present invention may be provided in a variety of antibody and antibody fragment formats and those which are specific for 3 R tau protein isoforms may be provided in any of the antibody formats set out herein. They may also have any of the levels of sequence variation set out herein.

### Epitopes

In one embodiment, an antibody or antigen-binding fragment thereof of the present invention binds to the same epitope, or at least substantially the same epitope, as one of the antibodies or antigen-binding fragments disclosed herein. The specific region or epitope of Tau can be identified by any suitable epitope mapping method known in the art in combination with any one of the antibodies provided by the present disclosure. Examples of such methods include screening peptides of varying lengths derived from tau protein for binding to the Tau-binding antibodies or binding fragments thereof of the present disclosure with the smallest fragment that can specifically bind to the antibody containing the sequence of the epitope recognized by the Tau-binding antibodies or binding fragments thereof. In one particularly preferred embodiment, residues forming part of the epitope of the antibody may be identified using mutational analysis, for instance by swapping residues of the tau protein for alanine and determining the impact on binding of the antibody or antigen-binding fragment thereof to tau protein, particularly 4 R tau protein.

In one embodiment at least one of K294, D295, N296, 1297, K298, and V300 may be present in the epitope of the antibody, in particular where those amino acid positions are defined relative to that of SEQ ID NO: 35.

In one particularly preferred embodiment, an antibody or antigen binding fragment is provided that binds to an epitope comprising at least one of the amino acid residues K294, D295, N296, and 1297. Preferably, the antibody or antigen-binding antibody is provided that binds to an epitope that comprises at least two of the amino acid residues K294, D295, N296, and 1297. More preferably, the antibody or antigen-binding fragment therefore binds to an epitope that comprises at least three of the amino acid residues K294, D295, N296, and 1297. In an even more preferred embodiment, the antibody or antigen-binding fragment thereof will bind to an epitope that comprises all four of the amino acid residues K294, D295, N296, and 1297.

In a further preferred embodiment, the antibody or antigen-binding fragment thereof may bind to such an epitope that further comprises amino acid residues K298 and V300 of tau protein. In one preferred embodiment, the antibody or antigen binding fragment thereof binds to an epitope comprising all of residues K294, D295, N296, 1297, K298, and V300 of tau protein.

In any of the above embodiments, the antibody or antigen-binding fragment thereof may be preferably a 4 R specific antibody. Hence, in one embodiment, the antibody or antigen-binding fragment thereof is a 4 R specific antibody that binds an epitope comprising K294, D295, N296, and 1297 of 4 R tau protein. In another preferred embodiment, the antibody or antigen-binding fragment thereof is a 4 R specific antibody that binds an epitope comprising K294, D296, N396, 1297, K298 and V300 of 4 R tau protein.

In one embodiment, the amino acids H299, P301, and G302 are not involved in the binding of the antibody or antigen-binding fragment thereof provided to tau protein.

In one embodiment, the antibody or antigen binding fragment thereof is a 4 R specific antibody that binds an epitope comprising K294, D296, N396, 1297, K298 and V300 of 4 R tau protein, but the H299, P301, and G302 residues are not involved in the binding of the antibody or antigen-binding fragment thereof to the tau protein.

In one embodiment, the overall epitope of the antibody or the antigen binding fragment thereof comprises amino acids in the region of residues K294 to V300 of tau 4 R protein. In one embodiment, amino acids for the epitope of the antibody or antigen-binding fragment thereof are only in that region. In another embodiment, residues forming the epitope are at least in that region.

Any of the antibodies or antigen binding fragments thereof mentioned can bind such epitopes. In one embodiment such antibodies may be chimeric, humanized or fully human monoclonal antibodies or can be used to obtain chimeric, humanized or fully human monoclonal antibodies. In one particularly preferred embodiment, the antibody or antigen-binding fragment thereof binding the epitope is a degrabody.

### Antibody Formats

An antibody or antigen-binding fragment of the present invention may be provided in any suitable format. In one preferred embodiment, an antibody or antigen-binding fragment thereof of the present invention may be an IgG class antibody or fragment thereof. In one embodiment, it may be an IgG1, IgG2, IgG3, or IgG4 isotype antibody and in particular IgG1. In another embodiment, an antibody of antigen-binding fragment thereof of the invention may also be an IgA, IgE, IgD, or IgM class antibody.

The present disclosure refers to antibodies and antibody fragments. Wherever mention is made of an antibody an antibody fragment may be employed unless specifically stated otherwise. Hence, antibodies of the present invention may comprise a complete antibody having full length heavy and light chains or be an antigen-binding fragment, for instance, a Fab, modified Fab, Fab′, modified Fab′, F(ab′)2, Fv, single domain antibody (e.g. VH or VL or VHH), scFv, bi, tri or tetra-valent antibody, Bis-scFv, diabody, triabody, tetrabody or epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities, e.g. bispecific or may be monospecific (see for example WO 92/22853, WO05/113605, WO2009/040562 and WO2010/035012). Examples of possible antibody formats are known in the art, for example as disclosed in the review “The coming of Age of Engineered Multivalent Antibodies”, Nunez-Prado et al Drug Discovery Today Vol 20 Number 5 Mar. 2015, page 588-594, D. Holmes, Nature Rev Drug Disc Nov 2011:10; 798, Chan and Carter, Nature Reviews Immunology vol. 10, May 2010, 301 incorporated herein by reference.

In one embodiment, antibody formats include those known in the art and those described herein, such as wherein the antibody molecule format is, or comprises, one of those selected from the group comprising or consisting of: diabody, BYbe, scdiabody, triabody, tribody, tetrabodies, TrYbe, tandem scFv, FabFv, Fab′Fv, FabdsFv, Fab-scFv, Fab-dsscFv, Fab-(dsscFv)2, diFab, diFab′, tandem scFv-Fc, scFv-Fc-scFv, scdiabody-Fc, scdiabody-CH₃, Ig-scFv, scFv-Ig, V-Ig, Ig-V, Duobody and DVDIg.

In one particularly preferred embodiment an antibody of the invention is a scFv-Ms-Fc format antibody.

In one embodiment, an antibody or antigen-binding fragment thereof of the invention is small enough to pass through the blood brain barrier (BBB).

In one embodiment, an antibody or an antigen-binding fragment thereof is a bispecific or multi-specific antibody where at least one of the antigen-binding sites is specific for 4 R tau protein isoforms or 3 R tau protein isoforms. Hence, in one preferred embodiment, it is such a bispecific antibody.

In a particularly preferred embodiment, an antibody or antigen-binding fragment thereof is one that can be expressed inside a cell and in particular is an intrabody. Intrabodies are antibodies, or antibody fragments, that are capable of expression, correct folding and antigen binding intracellularly. Intrabodies are typically capable of folding correctly in the reducing environment of the cytoplasm typically due to a lack of inter- and intra-chain disulphide bonds. Intrabodies are an alternative format for therapeutic antibodies of the present invention as in some instances they may be easier to target to cells than administration of the antibody itself.

In one embodiment, an antibody of the present invention is a targeted intrabody. For example, in one embodiment ER-intrabodies are targeted to the ER lumen using a KDEL or SEKDEL sequence on their C-terminus (as set out in Wheeler, Chen, and Sane 2003 Mol Ther, 8: 355-66; Lewis and Pelham 1992 Cell, 68: 353-64, both of which are incorporated by reference in their entireity). In one embodiment, an intrabody of the present invention is targeted to the cytoplasm (such a specific class of intrabody may be referred to as a cyto-intrabody) for example by the removal of a leader sequence. The intrabodies employed in the Examples of the present application are expressed in the cytoplasm of the cell. In another embodiment, an intrabody of the present invention is one targeted to the mitochondria or nucleus (for instance as set out in Biocca, Neuberger, and Cattaneo 1990 EMBO J, 9: 101-8, which is incorporated by reference in its entirety) with the addition of a suitable targeting signal.

IgG-derived intrabodies may be produced using the variable region domains of IgGs linked via a Gly₄Ser peptide linker to make a scFv fragment (Bird et al. 1988 Science, 242: 423-6 which is incorporated by reference). These can then be directly used as intrabodies as they no longer require disulphide bond formation for correct folding. In one embodiment, an intrabody of the present invention is a single domain antibody intrabody. For example, in one embodiment, an intrabody of the present invention is a heavy chain only antibody, for example Camelidae heavy chain only antibodies (HCabs) (Hamers-Casterman et al. 1993, Nature, 363: 446-8, which is incorporated by reference in its entirety). The single variable regions of HCabs (known as VHHs) can be used as intrabodies.

In one embodiment, an antibody or antigen-binding fragment of the present invention is a non-disulphide stabilised scFv and so may be used as an intrabody. In the scFv format the heavy and light chain variable regions may be physically linked by a flexible linker. That ensures the heavy and light chains pair correctly inside the cell and form a binding moiety without needing to form cysteine bridges between the heavy and light chains.

In one preferred embodiment, an antibody or antigen-binding fragment thereof provided may be one that comprises a degradation domain. Examples of possible degradation domains that may be employed include the C-terminal sequence of ornithine decarboxylase (ODC), human FkBP-12 protein (FkBP), the C-terminus of Hsc70 interacting protein (CHIP), the X-linked inhibitor of apoptosis protein (XIAP), von Hippel-Lindau protein (VHL), and the N-terminal of Simb protein (NSImb). The degradation domain may be one that is dependent on ubiquitin proteasomes for degradation in one embodiment. In another embodiment it is not so dependent. In one embodiment, the degradation domain is present as an N-terminal fusion to the rest of the antibody, for example a C-terminal fusion to a scFv. In another embodiment, it is an N-terminal fusion, for example an N-terminal fusion to a scFv.

In one embodiment, an antibody or antigen-binding fragment of the present invention is an intracellular degrading antibody, also known as a target degrading intrabody, or degrabody. In the degrabody format the intrabody is linked to a degradation domain and so effectively a degrabody is a specific kind of intrabody. Hence, in one particularly preferred embodiment of the present invention the antibody or antigen-binding fragment thereof is a degrabody.

In one embodiment, an antibody or antigen-binding fragment of the present invention is a rabbit antibody or fragment thereof. In another embodiment, an antibody or antigen-binding fragment of the present invention is one that comprises rabbit variable regions and the rest of the sequences of the antibody are mouse sequences. In another embodiment, the CDRs of the antibody are rabbit CDRs and the rest of the antibody is mouse. In a further embodiment, the antibody is a fully human antibody. In a further embodiment, all of the sequence of the antibody apart from the CDR sequences are human sequences.

### Variants

In one embodiment, rather than a specific sequence set out herein, an antibody or antibody fragment provided by the present invention may have a specific level of sequence identity or number of amino acid sequence changes compared to that specific sequence, so long as the antibody or fragment is still able to specifically bind whichever of 4 R or 3 R tau protein isoforms it is intended to be specific for. In another embodiment, a nucleic acid sequence may have a particular level of sequence identity compared to one of the specific sequences set out herein, provided that it still encodes an antibody or fragment thereof, or a constituent of those, which can still bind specifically to whichever of 4 R or 3 R tau protein isoforms it is intended to be specific for.

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

Hence, the present disclosure extends to novel polypeptide sequences disclosed herein and sequences at least 80% similar or identical thereto, for example 85% or greater, such 90% or greater, in particular 95%, 96%, 97%, 98% or 99% or greater in similarity or identity. “Identity”, as used herein, indicates that, at any particular position in the aligned sequences, the amino acid residue is identical between the sequences. In one embodiment, a sequence may have one of those levels of sequence identity provided that the encoded antibody or fragment is still able to specifically bind 4 R or 3 R tau protein isoforms.

A particular amino acid sequence may differ from one of the specific amino acid sequences set out herein by up to 10, 9, 8, 7, 6, 5, 4, 3, 2, or 1 amino acid sequence changes, provided that it is still able to specifically bind 4 R or 3 R tau protein isoforms. In one embodiment, it may differ from the specific sequence by that number of sequence changes where the sequence changes are conservative ones.

As well as variant sequences defined by percentage identity or number of sequence changes, the present invention further provides an antibody or antigen-binding fragment defined by its ability to cross-block one of the specific antibodies or fragments set out herein. It may be that the antibody also has one of the recited levels of sequence identity or number of sequence changes as well. Cross-blocking antibodies can be identified using any suitable method in the art, for example by using competition ELISA or BIAcore assays where binding of the cross blocking antibody to antigen (the particular tau protein of interest, so for instance 4 R or 3 R, or one of the peptides from those peptides discussed herein) prevents the binding of an antibody or antibody fragment of the present invention or vice versa. In one embodiment, the antibody produces at least 50%, 60%, 70%, 80%, 90% or more reduction of binding of the specific antibody or antigen-binding fragment disclosed herein.

### Peptides

The present invention also provides peptides that may be used to generate antibodies and antigen-binding fragments of such antibodies. In one embodiment, a peptide comprising the sequence bridging exons 9 and 10 of tau protein is provided. For example, in one embodiment, a peptide comprising, or consisting of, the sequence of SEQ ID NO: 36 is provided, also known as the TE9/11 peptide. In another embodiment, the present invention provides a peptide sequence that corresponds to the amino acid sequence encoded by exon 10 of tau as that means the antibody is likely to recognise 4 R tau protein isoforms specifically. In one embodiment, the peptide does not comprise an amino acid that represents a post-translational modification site in native 4 R tau protein isoforms. In one embodiment, a peptide comprising, or consisting of, the amino acid sequence of SEQ ID NO: 37 is provided, which is also referred to as the TE10 peptide.

In one embodiment, the peptide comprises further amino acid residues that do not originate from a tau protein, for example it may comprise a terminal cysteine to help conjugate the peptide to a carrier. In one embodiment, the peptide is conjugated to a carrier. For example, in one embodiment the carrier protein is KLH, ovalbumin, or BSA. In one embodiment, the peptide is conjugated as a linear peptide to a carrier and in particular where the portion of the peptide most likely to give rise to specific antibodies is further away from the carrier. In another embodiment, the peptide is a cyclic peptide, for example one joined to a carrier so that the amino acid residues most likely to give rise to an antibody specific for 4 R or 3 R tau protein isoforms are further away from the carrier.

In one embodiment, an antibody or antigen-binding fragment thereof which is obtainable by immunizing an animal with one of the above-mentioned peptides, in particular one conjugated to a carrier, is provided. In one embodiment, the antibody or antibody fragment is one obtained via such a method. In one embodiment, the immunized animal is a rabbit. Any suitable method may be used to identify the desired antibodies from such an immunized animal, for example screening via ELISA using tau peptides or proteins or any of the other methods discussed herein may be employed.

In one embodiment, an antibody or antigen-binding fragment of the present invention specific for 3 R tau protein isoforms is not an antibody referred to in de Silva et al (2003) Neuropathology and Applied Neurobiology, 29: 288-302. In one embodiment, the antibody or antigen-fragment is not the RD3 antibody disclosed in de Silva et al. In another embodiment, the antibody or antigen-binding fragment thereof is specific for 4 R tau protein isoforms, but is not an antibody referred to in Croft et al (2018) https://doi.org/10.1371/journal.pone.0195211. In one embodiment, the antibody or antigen-binding fragment thereof is specific for 4 R tau protein isoforms but is not an antibody referred to in either of de Silva et al (2013) and Croft et al (2018).

### Nucleic Acids, Vectors, and Host Cells

In a further aspect, there is provided a nucleotide sequence, for example a DNA sequence encoding an antibody or antibody fragment of the present invention as described herein. In one embodiment, there is provided a nucleotide sequence, for example a DNA sequence encoding an antibody or fragment of the present invention as described herein. In one embodiment, the nucleotide sequence is collectively present on more than one polynucleotide but the nucleotide sequences together are able to encode an antibody or antibody fragment of the present invention.

The invention herein also extends to a vector comprising a nucleotide sequence as defined above. The term “vector” as used herein refers to a nucleic acid molecule capable of transporting another nucleic acid to which it has been linked. An example of a vector is a “plasmid,” which is a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell, where they are subsequently replicated along with the host genome. In the present specification, the terms “plasmid” and “vector” may be used interchangeably as a plasmid is the most commonly used form of vector. General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing. The term vector herein also includes, for example, particles comprising the vector, for example LNP (Lipid Nanoparticle) particles and in particular LNP-mRNA particles. It also includes viral particles used for transferring a vector of the present invention.

A vector of the present invention may include a selectable marker. The term “selectable marker” as used herein refers to a protein whose expression allows one to identify cells that have been transformed or transfected with a vector containing the marker gene. A wide range of selection markers are known in the art. For example, typically the selectable marker gene confers resistance to drugs, such as G418, hygromycin or methotrexate, on a host cell into which the vector has been introduced. The selectable marker can also be a visually identifiable marker such as a fluorescent marker for example. Examples of fluorescent markers include rhodamine, FITC, TRITC, Alexa Fluors and various conjugates thereof. In one embodiment, the selectable marker may be flanked by sequences that allow removal of the marker, for example loxP or frt.

Also provided is a host cell comprising one or more cloning or expression vectors comprising one or more DNA sequences encoding an antibody or antigen-binding fragment thereof of the present invention. Any suitable host cell/vector system may be used for expression of the DNA sequences encoding the antibody or fragment of the present invention. Bacterial, for example E. coli, and other microbial systems may be used or eukaryotic, for example mammalian, host cell expression systems may also be used. Suitable mammalian host cells include CHO, myeloma or hybridoma cells. A host cell comprising a nucleic acid molecule or vector of the present invention is also provided.

### Detection of Tau and Diagnosis

The antibodies and antigen-binding fragments thereof of the present invention may be used in diagnosis/detection kits. In one embodiment, antibodies or antibody fragments of the present invention are fixed on a solid surface. The solid surface may for example be a chip, or an ELISA plate. The binding molecules, in particular antibodies, of the present invention may be for example conjugated to a fluorescent marker which facilitates the detection of bound antibody-antigen complexes. They can be used for immunofluorescence microscopy. Alternatively, an antibody or antigen-binding fragment thereof, may also be used for western blotting or ELISA.

In one particularly preferred embodiment, the ability to bind 3 R or 4 R tau protein isoforms is measured where the tau protein is present in the same form that it is physiologically, for example not in denatured form. In one preferred embodiment it is present in a cell. In one embodiment, the antibody or antigen-binding fragment thereof may bind the protein when it is present on a cell and hence be used to detect the protein when present on the cell. In one embodiment, the antibody or antigen-binding fragment thereof may bind the protein when it is present in the extracellular space, such as in the form of secreted fibrils.

Any suitable method may be employed to determine the binding of a given antibody or antigen-binding fragment thereof to 4 R tau compared to 3 R tau. In one embodiment, an ELISA is employed: for example, binding to immobilised tau 4 R protein may be compared to that shown by immobilized 3 R tau protein. Binding to the peptides discussed herein may also be measured, and compared, by ELISA.

In another embodiment, western blotting may be employed to detect binding of a given antibody or antigen-fragment thereof to 4 R and 3 R tau protein isoforms. Such western blots may be performed on any suitable material, for example on cell lysates of cells known to express 4 R or 3 R or both tau protein isoforms. In one embodiment, the cells employed are iPSc cells and in particular such cells which are known to express a particular tau protein. In another embodiment, they are neurones differentiated from iPSCs. In one embodiment, Western blots are performed on tissue samples, for example on brain tissue from a subject. In one preferred embodiment, a Peggy Sue Simple Western is employed.

In another embodiment, the ability of an antibody or antigen-binding fragment thereof to bind a particular form of tau protein is measured by immunofluorescence. For example, the antibody or antigen-binding fragment thereof may be conjugated to a fluorochrome itself and the present invention also provides such conjugated antibodies or antigen-binding fragments thereof. Alternatively, binding of an antibody or antigen-binding fragment thereof to a tau protein may be identified, and measured, using a secondary antibody, for example specific for the species of the primary antibody. In one embodiment such immunofluorescence is performed on cells expressing 4 R or 3 R tau protein, for example CHO cells, in particular CHOK1 cells over-expressing the desired tau. In another embodiment, immunohistochemistry is performed on iPSc cells. In another embodiment, binding may be measured on a tissue sample, for example via immunohistochemistry (IHC) by fluorescence or other means.

In one particularly preferred embodiment, iPSCs (Induced Pluripotent Stem cells) are used to assess an antibody or antigen-binding fragment of the present invention. In another preferred embodiment, neurons are employed, for example neurons obtained by differentiating iPSCs. In one preferred embodiment, cells are isolated from a subject with a particular disorder, such as any of those mentioned herein and used to assess an antibody or antigen-binding fragment thereof of the present invention. In one embodiment, fibroblasts are isolated from a subject with a tauopathy and used to produce iPSCs, then neurons. In one embodiment, the subject used to obtain the cells from is one with a mutation in the tau gene (MAPT) gene, for instance one of the mutations mentioned herein. For example, fibroblasts from such subjects may be used to generate iPSCs which will comprise the same mutation in the tau gene as the subject. In a particularly preferred embodiment, such iPSCs are differentiated into neurons. In one embodiment, the neurons are cultured for at least five, six, seven, eight, or nine months before being used. In one embodiment, a comparison may be made with a control cell line that lacks any mutation associated with a tauopathy in the tau gene, for example cells isolated from a healthy subject lacking such mutations. In one embodiment, fibroblasts are isolated from a healthy control subject, used to generate iPSCs, and then differentiated into neurons.

In another embodiment, rather than use cells from a subject with a specific disease mutation in the tau gene, mutations associated with a tauopathy are engineered into a chosen cell line, for example one which is, or is used to generate, iPSCs. In one embodiment, the iPSCs are then differentiated into neurons comprising the mutation(s).

In one embodiment, an antibody or antigen-binding fragment of the present invention is used to detect 4 R tau protein isoforms or 3 R tau protein isoforms. For example, the present invention provides a method of detecting or measuring 4 R tau protein isoforms comprising: (a) contacting a test sample with an antibody or antigen binding fragment thereof of the present invention; and (b) detecting any binding of the antibody. Any of the detection means discussed herein may be employed, for example ELISA, immunofluorescence, flow cytometry analysis or immunohistochemistry may be used as the detection means. In one embodiment, the test sample comprises a cell lysate, or cells, or tissue, for example from a subject. In one embodiment, the test sample is one comprising neurones or a lysate from them. In one embodiment, the test sample is brain tissue or lysate. In one embodiment, such a method may also comprise a positive control, for example one known to express 4 R tau protein or which expresses it at normal levels. Such methods may also comprise performing the method separately with an antibody that binds all tau protein isoforms of the subject to give an indication of the amount of 4 R tau protein compared to that of the total amount of tau protein present.

In another embodiment, rather than an antibody or antigen-binding fragment thereof of the present invention specific for 4 R tau protein isoforms, one specific for 3 R tau protein isoforms is employed in a method as set out above for 4 R tau protein isoforms, except the method is for detection or measurement of 3 R tau protein isoforms.

The method may comprise analysing a test sample both for the level of 4 R tau protein isoforms and for the level of 3 R tau protein isoforms. For example, the method may comprise testing two portions of the same test material or staining simultaneously with both an antibody or fragment thereof specific for 4 R tau protein isoforms and an antibody or fragment thereof specific for 3 R tau protein isoforms. In one example, each is differently labelled, for example with different fluorochromes to allow a direct comparison of the relative amounts of 4 R tau protein and 3 R tau protein.

Such a method for detecting the relative amounts of 4 R tau protein isoforms and 3 R tau protein isoforms may be used to detect an imbalance of the two, for example compared to a sample from a healthy subject or a sample from the same subject before they developed a tauopathy. In one embodiment an antibody or fragment thereof of the present invention may be used to diagnose a tauopathy. In one preferred embodiment, an antibody or fragment thereof of the present invention is used to diagnose a condition involving an imbalance of 4 R and 3 R tau protein isoforms. For example, frontotemporal dementia and parkinsonism's linked to chromosome 17 (FTDP-17), progressive supranuclear palsy (PSP), and corticobasal degeneration (CBD) are all thought to involve 4 R tau protein isoforms predominating and so the invention may be used to detect such an imbalance as a way to diagnose the condition. Conversely, Pick's Disease is considered to be one where there is more 3 R tau protein than 4 R tau protein. Hence, again the present invention may be used to diagnosis that condition based on the identification of such an imbalance.

In one preferred embodiment, an antibody or antigen-binding fragment thereof is one which is able to specifically bind 4 R tau protein isoforms but not 3 R tau protein isoforms in a Peggy Sue Simple Western. In another it is able to show such specificity as measured by flow cytometry, in particular on cells over-expressing tau. In another embodiment, it shows such specificity when used to analyse a human brain sample, for instance by Western blot. In a particularly preferred embodiment, it will show such specificity when immunofluorescence is performed using the antibody or fragments on neurones, particularly those obtained from iPSC expressing the relevant tau protein at physiological levels.

In one preferred embodiment, an antibody or antigen-binding fragment thereof is one which is able to specifically bind 3 R tau protein isoforms but not 4 R tau protein isoforms in a Peggy Sue Simple Western blot. In another it is able to show such specificity as measured by flow cytometry, in particular on cells over-expressing tau. In another embodiment, it shows such specificity when used to analyse a human brain sample, for instance by Western blot. In a particularly preferred embodiment, it will show such specificity when immunofluorescence is performed using the antibody or fragments on neurones, particularly those obtained from iPSC expressing the relevant tau protein at physiological levels.

Pathological conditions

In one embodiment, an antibody or antigen-binding fragment thereof may be used to treat or diagnose a tauopathy. In one embodiment, the condition to be treated is a primary tauopathy. In one embodiment, the condition may involve frontotemporal lobar degeneration (FTLD): for example, it may be characterised by neuronal tau inclusions with predominant cell death in the frontal and temporal lobes of the brain. In another embodiment, the condition may be a secondary tauopathy, so a condition where tau pathology is observed in association with other brain pathologies.

The present invention may be used to treat or diagnose tauopathies in general, for example, Alzheimer's Disease (AD) and a range of other conditions, including progressive supranuclear palsy (PSP), corticobasal degeneration (CBD), Pick's disease, or frontotemporal dementia (FTD).

In one preferred embodiment of the present invention, the condition to be treated or diagnosed is Alzheimer's Disease (AD), which is a secondary tauopathy.

In one embodiment, an antibody or antigen binding fragment thereof of the present invention is used to shift the balance of 3 R and 4 R tau protein in a subject with a tauopathy. For example, an antibody specific for 3 R tau protein may be used to shift the balance more towards 4 R tau protein. Alternatively, an antibody specific for 4 R tau protein may be used to shift the balance more towards 3 R tau protein.

In one preferred embodiment, the condition to be treated or diagnosed via the present invention may be Pick's Disease (PD). In one preferred embodiment, an antibody or antigen-fragment thereof of the present invention specific for 3 R tau protein isoforms may be used to treat Pick's Disease. In a preferred embodiment, the antibody or antigen-binding fragment thereof may be used to change the balance away from the 3 R tau protein isoforms. For example, applying the present invention in that way may result in less or eliminate Pick bodies. Employing an antibody of the present invention specific for 3 R tau protein isoforms may eliminate, reduce or stabilise neuronal and glial loss in the frontal, temporal and parietal lobes of the brain in a subject and in particular a subject with Pick's Disease. In one embodiment, treating a subject with an antibody or antigen-binding fragment thereof of the present invention specific for 3 R tau protein isoforms may shift the ratio of 3 R:4 R tau proteins isoforms in the subject towards, or to, the level seen in individuals who does not have Pick's Disease.

In another embodiment, an antibody or antigen-binding fragment thereof of the present invention is used to treat a condition where there is more 4 R tau protein than in a healthy subject and in particular one where the ratio of 4 R:3 R tau protein is shifted towards 4 R tau protein. Hence, an antibody or antigen-binding fragment of the present invention specific for 4 R tau protein may be used to treat a 4 R tauopathy. Examples of such conditions that may be treated include: Frontotemporal dementia and parkinsonism's linked to chromosome 17 (FTDP-17); progressive supranuclear palsy (PSP); and corticobasal degeneration (CBD).

In one embodiment, the subject to be treated may have a MAPT mutation, i.e. a mutation in the tau gene. In one embodiment, the subject may have a splice mutation in or near intron 10 which results in overinclusion of exon10 (MTBR R3) and an increase in 4 R tau. In one preferred embodiment the tauopathy involves a 10+16 mutation. In other words, in one embodiment the mutation is a IVS 10+16 C-T mutation. In one embodiment, the subject has a MAPT mutation that results in an increase in the mRNA for 4 R tau protein. In one embodiment, an antibody or antigen-binding fragment thereof of the present invention specific for 4 R tau protein may be used to treat such a condition. In one embodiment, the subject to be treated has FTDP-17 and in particular early onset FTDP-17.

The present invention may be applied to treat, prevent or diagnose tauopathies stemming from other mutations that can affect the inclusion of exon 10 and hence result in an increase in 4 R tau mRNA. These can be within the stem loop structure such as S305N and S305I which in a similar way to the intronic mutations destabilise the stem loop causing increased inclusion of exon 10 (Hasegawa et al. 1998, FEBS letters, 443 (2), 93-96; Kovacs et al., 2008, protein-based neuropathology and molecular classification of human neurodegenerative diseases: 251-272; Stanford et al., 2000, Brain, 123(5), 857-859).

Alternatively, mutations within regulatory elements of exon 10 have been demonstrated to increase exon 10 inclusion. The mutations N279K and L284L strengthen enhancer regions within exon 10 resulting in an increased inclusion and excess 4 R tau expression (D'Souza and Schellenberg, 2002., Journal of Biological Chemistry, 277 (29), 26587-99; D'Souza et al., 1999, Proceedings of the National Academy of Sciences, 96 (10), 5598-603; Hasegawa et al., 1999, supra). Other mutations within exon 10 have been demonstrated to increase 4 R tau expression include N296N and N296H which have been postulated to either disrupt silencer regions (D'Souza and Schellenberg 2002, supra) or create a new enhancer (Andrew Grover et al.,2002, Neuroscience letters, 323 (1), 33-36). Somewhat counterintuitively mutations within exons 12 and 13 (E342V and N410H respectively) have been reported to increase the expression of 4 R tau within neurons (Lippa et al. 2000, Annals of neurology, 48 (6), 850-58). Again, the present invention may be employed to treat tauopathies involving such mutations.

Mitochondrial impairment can also be a feature of taupoathies. In one embodiment, treatment with an antibody or antigen-binding fragment thereof of the present invention may help restore mitochondrial function in a tauopathy. For example, in such tauopathies mitochondrial membrane potential may be affected, and treatment with an antibody or antigen-binding fragment thereof may help restore mitochondrial membrane potential to, or towards, normal levels. In one embodiment, the subject to be treated is one that displays mitochondrial dysfunction. In one embodiment, the subject may be one who displays altered mitochondrial membrane potential. In one embodiment, the subject displays lowered membrane potential. In a preferred embodiment, the subject displays raised membrane potential and in particular displays such raised membrane potential and has a 10+16 MAPT mutation. In one embodiment, such a subject may display excess 4 R tau protein isoforms. In another embodiment, the subject has a P301L mutation and in particular may display hyperpolarised mitochondrial membranes. In one embodiment, employing the invention may mean that mitochondrial membrane potential may return, or at least be closer to, normal.

In one particularly preferred embodiment, antibodies or antigen-binding fragments thereof that are able to cross the blood brain barrier may be employed. In a further preferred embodiment, rather than administering an antibody or antigen-binding fragment itself, a nucleic acid or nucleic acids or vector or vectors of the present invention may be administered, in one preferred embodiment ones encoding an intrabody or degrabody may be administered to a subject. In another embodiment, host cells of the present invention able to express an antibody or antigen-binding fragment of the present invention are employed.

### Pharmaceutical Compositions

In one aspect, there is provided a pharmaceutical composition comprising: (a) an antibody or antigen-binding fragment thereof, a nucleic acid molecule or molecules, or a vector or vectors of the present invention; and (b) a pharmaceutically acceptable carrier or diluent. In one preferred embodiment, the pharmaceutical composition comprises an antibody or antigen-binding fragment. The composition may be in solid, or liquid form and may be, inter alia, be in the form of a powder, a tablet, a solution or an aerosol.

Also provided is an antibody or antigen-binding fragment thereof, a nucleic acid molecule or molecules, a vector or vectors, or a pharmaceutical composition of the present invention for use in a method of treatment or diagnosis of the human or animal body. Further provided is the use of an antibody or antigen-binding fragment thereof, a nucleic acid molecule or molecules, a vector or vectors, or a pharmaceutical composition of the present invention for the manufacture of a medicament for the treatment of a pathological condition or disorder. In one embodiment where a therapeutic of the invention is administered to a subject who is also being given a second therapeutic agent, the two may be given, for example, simultaneously, sequentially or separately. In one embodiment, the two are given in the same pharmaceutical composition. In another embodiment, the two are given in separate pharmaceutical compositions.

A composition of the present invention will usually be supplied as a sterile, pharmaceutical composition. A pharmaceutical composition of the present invention may additionally comprise a pharmaceutically acceptable adjuvant. In another embodiment, no such adjuvant is present in a composition of the present invention. The present invention also provides a process for preparation of a pharmaceutical or diagnostic composition comprising adding and mixing the binding molecule, in particular antibody, of the present invention together with one or more of a pharmaceutically acceptable excipient, diluent or carrier.

The term “pharmaceutically acceptable excipient” as used herein refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes. This may include production and sterilization by filtration of the buffered solvent solution used for the formulation, aseptic suspension of the antibody in the sterile buffered solvent solution, and dispensing of the formulation into sterile receptacles by methods familiar to those of ordinary skill in the art.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates. Pharmaceutically acceptable carriers in therapeutic compositions may additionally contain liquids such as water, saline, glycerol and ethanol. Such carriers enable the pharmaceutical compositions to be formulated as tablets, pills, dragées, capsules, liquids, gels, syrups, slurries and suspensions, for ingestion by the patient.

A thorough discussion of pharmaceutically acceptable carriers is available in Remington's Pharmaceutical Sciences (Mack Publishing Company, N.J. 1991).

The term “therapeutically effective amount” as used herein refers to an amount of a therapeutic agent needed to treat, ameliorate or prevent a targeted disease or condition, or to exhibit a detectable therapeutic or preventative effect. For any binding molecule, in particular antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans. The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 50 mg/kg, for example 0.1 mg/kg to 20 mg/kg per day. Alternatively, the dose may be 1 to 500 mg per day, such as 10 to 100, 200, 300 or 400 mg per day. Pharmaceutical compositions may be conveniently presented in unit dose forms containing a predetermined amount of an active agent of the invention. In one embodiment, the amount in a given dose is at least enough to bring about a particular function.

Any of a number of routes of administration may be employed to administer the present invention to a subject including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Direct delivery of the compositions will generally be accomplished by injection, subcutaneously, intraperitoneally, intravenously or intramuscularly, or delivered to the interstitial space of a tissue. The compositions can also be administered into a specific tissue of interest. In one embodiment, the administration is to the brain. Dosage treatment may be a single dose schedule or a multiple dose schedule. Where the product is for injection or infusion, it may take the form of a suspension, solution or emulsion in an oily or aqueous vehicle and it may contain formulatory agents, such as suspending, preservative, stabilising and/or dispersing agents. Alternatively, a pharmaceutical composition of the present invention may be in dry form, for reconstitution before use with an appropriate sterile liquid. If the composition is to be administered by a route using the gastrointestinal tract, the composition may contain agents which protect the antibody from degradation but which release the antibody or antigen-binding fragment thereof once it has been absorbed from the gastrointestinal tract. A nebulisable formulation according to the present invention may be provided, for example, as single dose units (e.g., sealed plastic containers or vials) packed in foil envelopes. Each vial contains a unit dose in a volume, e.g., 2 ml, of solvent/solution buffer.

In one embodiment, an antibody or antigen-binding fragment, nucleic acid, vector, host cell, or pharmaceutical composition of the present invention is administered to the brain or in such a way that it is able to gain access to the brain.

The present invention also provides a process for preparation of a pharmaceutical or diagnostic composition comprising adding and mixing an antibody or antigen-binding fragment thereof of the present invention, in particular an antibody, together with one or more of a pharmaceutically acceptable excipient, diluent or carrier. The antibody or antigen-binding molecule of the present invention may be the sole active ingredient in the pharmaceutical or diagnostic composition or may be accompanied by other active ingredients including antibody ingredients or non-antibody ingredients such as steroids or other drug molecules. The pharmaceutical compositions suitably comprise a therapeutically effective amount of the antibody or antigen-binding fragment thereof. The term “therapeutically effective amount” as used herein refers to an amount of a therapeutic agent needed to treat, ameliorate or prevent a targeted disease or condition, or to exhibit a detectable therapeutic or preventative effect.

Pharmaceutical compositions may be conveniently presented in unit dose forms containing a predetermined amount of an active agent of the invention per dose. A pharmaceutical composition of the present invention may be provided in a receptacle that provides means for administration to a subject. A pharmaceutical composition of the present invention may be provided in a prefilled syringe. The present invention therefore provides such a loaded syringe. It also provides an auto-injector loaded with a pharmaceutical composition of the present invention.

It is also envisaged that the binding molecule, in particular antibody, of the present invention may be administered by use of gene therapy. In order to achieve this, where the binding molecule is an antibody, DNA sequences encoding the heavy and light chains of the antibody molecule under the control of appropriate DNA components are introduced into a patient such that the antibody chains are expressed from the DNA sequences and assembled in situ. In another particular preferred embodiment, the sequences encoding an intrabody of the present invention may be administered to a subject.

The present invention also extends to a kit, comprising an antibody or antigen-binding fragment thereof of the invention, optionally with instructions for administration. In yet another embodiment, the kit further comprises one or more reagents for performing one or more assay or method, such as those discussed herein.

In one embodiment, molecules of the present invention including an antibody or antigen-binding fragment thereof of the invention is provided for use as a laboratory reagent.

“Purified form” as used supra is intended to refer to at least 90% purity, such as 91, 92, 93, 94, 95, 96, 97, 98, 99% w/w or more pure.

In the context of this specification “comprising” is to be interpreted as “including”. Aspects of the invention comprising certain elements are also intended to extend to alternative embodiments “consisting” or “consisting essentially” of the relevant elements.

Positively recited embodiments may be employed herein as a basis for a disclaimer.

Where the singular is referred to herein, the plural is also encompassed unless otherwise stated or apparent. In particular, the singular forms “a,” “an,” “the” and the like include plural referents unless the context clearly dictates otherwise.

All references referred to herein are specifically incorporated by reference.

The sub-headings herein are employed to assist in structuring the specification and are not intended to be used to construct the meaning of technical terms herein.

Sequences of the invention are provided herein below.

### EXAMPLE

### Example 1—Peptide Immunogen Design 3 R Tau

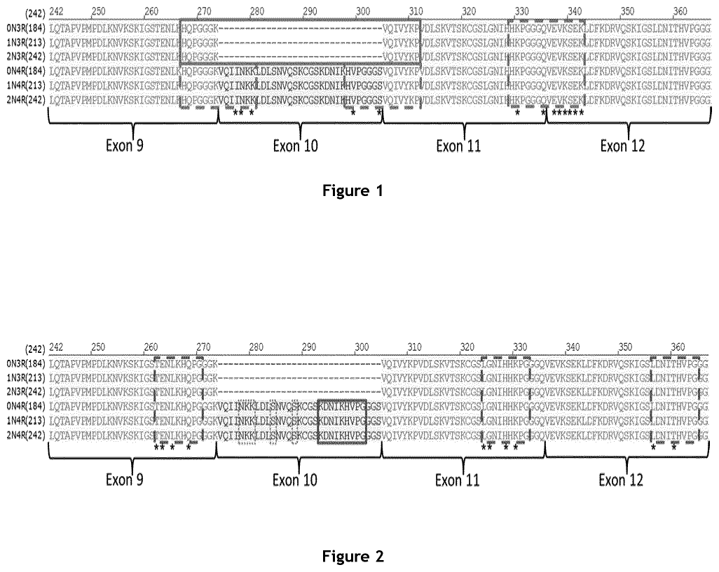

The amino acid sequence of the tau MTBR, exons 9-12 is shown in FIG. 1. This splicing of exon 10 creates a unique exon boundary between exons 9 and 11 which only found in 3 R tau. This epitope was used as a way to raise an antibody that would confer specificity for 3 R tau over 4 R tau. To drive an immune response to this epitope, a peptide was designed to cover amino acids 268-311 of tau (the peptide is named TE9/11), as denoted by the solid box in FIG. 1 and the TE9/11 sequences in Table 1.

TABLE 1
Sequence alignment of TE9/11 with potentially cross-
reactive epitopes present on all tau isoforms

| AA number | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| TE9/11 | H | Q | P | G | G | G | K | V | Q | I | V | Y | K | P |
| Cross-reactive - | H | V* | P | G | G | G | S* | V | Q | I | V | Y | K | P |
| E10/11 | | | | | | | | | | | | | | |
| Cross-reactive - | H | Q | P | G | G | G | K | V | Q | I | I* | N* | K | K* |
| E9/10 | | | | | | | | | | | | | | |
| Cross-reactive - | H | K* | P | G | G | G | Q* | V | E* | V* | K* | S* | E* | K* |
| E11/12 | | | | | | | | | | | | | | |

TE9/11 shares homology to other regions of tau that exist at the exon boundaries between the all exons of the tau MTBR. These potentially cross-reactive epitopes are highlighted in the dashed boxes (FIG. 1), with the asterisk indicating sequence difference. For a clearer comparison Table 1 shows an exact peptide alignment of the potential cross-reactive epitope, with the asterisk indicating sequence differences.

The first of these is the exon boundary between exon 10 and 11 (tau residues 299-312 FIG. 1)) which differs from TE9/11 by only two amino acids (peptide residues 2 and 7 (Table 1)). The second region of similarity exists at the boundary between exon 9 and 10 (tau residues 268-281 (FIG. 1)), which differs by three amino acids (peptide residues 11, 12 and 14 (Table 1)). Finally, there is sequence homology at the site of the boundary between exons 11 and 12 (tau residues 330-343 (FIG. 1)). However, there are 8 differences (peptide residues 2,7 and 9-14 (Table 1), representing 50% of the epitope space so this is less likely to drive non-specific antibody binding.

### Example 2—Peptide Immunogen Design 4 R Tau

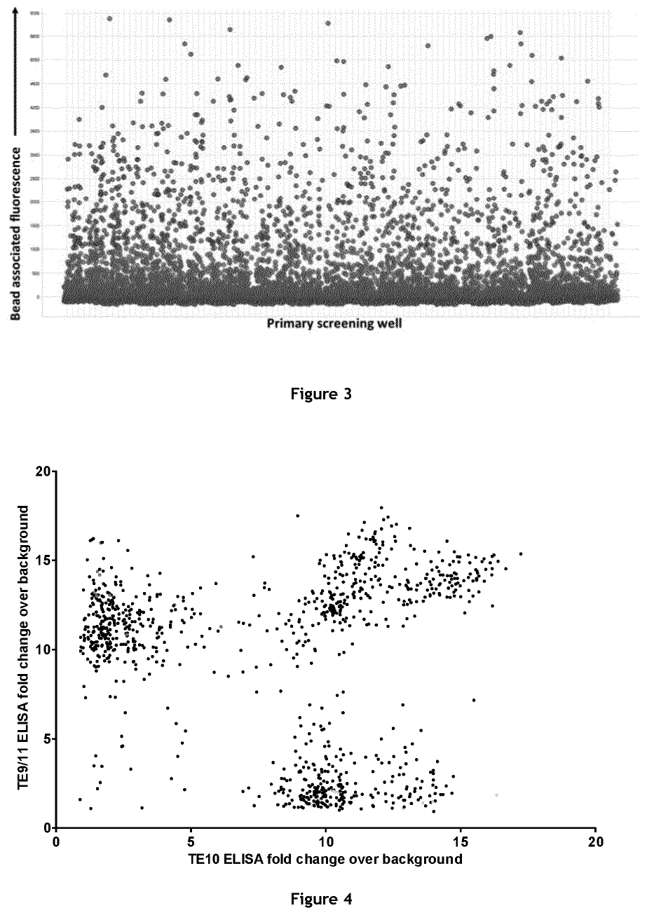

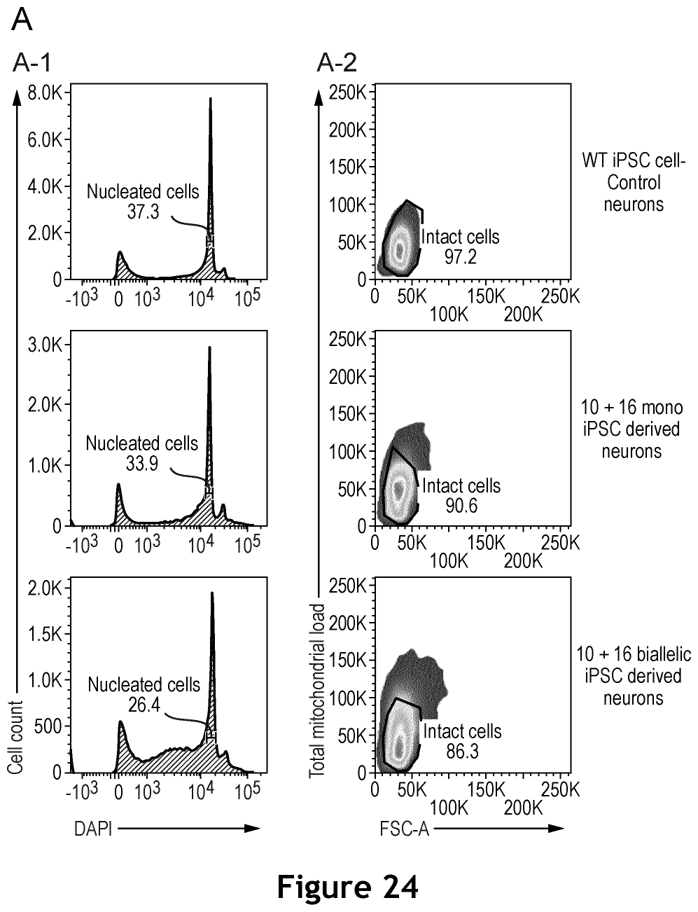

As previously discussed, the unique feature of 4 R tau over 3 R tau is the presence of exon 10 in 4 R tau. This represents an epitope for an antibody that would give specificity to 4 R tau over 3 R tau. It is worth noting that it might be possible to achieve 4 R specificity by targeting the exon boundaries between Exons 9/10 and 10/11. In both cases there would only be one amino acid to target (I278 or S301 respectively). It is also important to consider there are several sites of known post translational moderation within exon 10, N279, K280/281 and S285/289) (Ercan et al. 2017, Mol Neurodegener, 12 (1), 87; Kontaxi et al. 2017, Front Mol Biosci, 4, 56; Mair et al. 2016, Anal Chem, 88 (7), 3704-14) (shown in FIG. 2 in the dashed box within exon 10). To ensure an antibody would be specific to 4 R tau, and bind regardless of the post translational modification state of tau, peptide TE10 was designed to cover tau amino acids 294-302, (FIG. 22), as this is within exon 10 and in a region of exon 10 and avoids the post translational modification sites. FIG. 2 shows the location of TE10 in the solid box within the 4 exons of the tau MTBR. The TE10 line in Table 2 shows the exact peptide sequence.

TABLE 2
Sequence alignment of TE10 peptide with potential
cross-reactive sites in Exons 9, 11 and 12 of tau.

| AA number | 1 | 2 | 3 | 4 | 5 | 6 | 7 | 8 | 9 |
|---|---|---|---|---|---|---|---|---|---|
| TE10 | K | D | N | I | K | H | V | P | G |
| Cross reactive- | L* | D | N | I | T* | H | V | P | G |
| E12 | | | | | | | | | |
| Cross reactive- | L* | G* | N | I | H* | H | K* | P | G |
| E11 | | | | | | | | | |
| Cross reactive- | T* | E* | N | L* | K | H | Q* | P | G |
| E9 | | | | | | | | | |

Given the repetitive nature if the MTBR, TE10 shares sequence homology to three other exons of the MTBR (highlighted in the dashed boxes in FIG. 2 and aligned in Table 2). The first of these is within exon 12 (tau residues 357-365 (FIG. 2)) which differs from the TE10 peptide by two amino acids (peptide residue 1 and 5 (Table 2)). The second region of similarity exists within exon 9 (tau residues 263-271 (FIG. 2)) which differs by four amino acids (peptide residue 1, 2, 5 and 7 (Table 2)). Finally, there is sequence homology within exon 11 (tau residues 325-333 (FIG. 2)) which differs by four amino acids (peptide residue 1, 2, 4 and 7 (Table 2)). In Table 2, the differing amino acids are indicated with asterisks.

### Example 3—Peptide Conjugation for Immunisation

To elicit an immune response, the peptides were conjugated to the carrier proteins keyhole limpet hemocyanin (KLH), ovalbumin (OVA) and bovine serum albumin (BSA). All peptides were also conjugated to biotin to allow for screening via streptavidin capture to beads or plates (carried out by CRO Peptide Synthetics).

Throughout an immunisation the immunodominant position of a peptide immunogen is located furthest from the carrier protein, as this is most exposed to the immune system. Therefore TE9/11 was conjugated as cyclic a peptide to ensure the predicted key residues 2, 7, 11 and 12 (Table 2), were presented in the most immunodominant positions. TE10 (designed to confer a 4 R tau specific immune response) was designed as linear peptide coupled to the carrier protein via its C-terminus. This was to ensure the lysine residues at positions 1 and 5 of the peptide, the only 2 amino acids that are unique to TE10 (Table 2), were in the immunodominant position. For both peptides, a single cysteine was added to the peptide for linkage via the thiol group to the maleimide linker and carrier protein or biotin-PEG-maleimide. Cyclisation of the peptides was achieved via N to C-terminal amide bond formation to generate a cyclic peptide loop.

### Example 4—3 R and 4 R Specific Antibody Identification from Rabbits

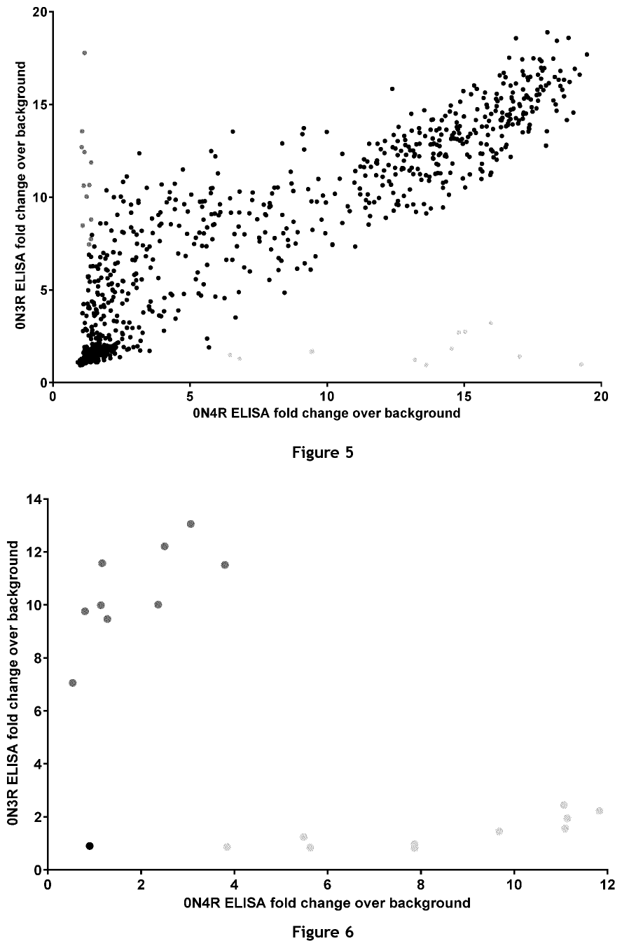

The conjugated peptides were used to immunize rabbits. B-cells from rabbit 6170 and 6171 were activated into antibody secreting B-cells in a 400×96 well plate culture. Culture supernatants were primary screened for binding. This homogenous fluorescence assay was to a mix of the biotinylated TE9/11 and TE10 peptides captured onto streptavidin beads. FIG. 3 shows the primary screening data from the 50×384 well assay screening plates. Highlighted in green are the wells where bead associated fluorescence was observed, indicating antibodies with specificity to either TE10 or TE9/11. Effectively, the colonies highlighted green are roughly above a bead associated fluorescence of 1000 in the graph. These hits were selected based on a binding value threshold of 1000 bead associated fluorescent units. This threshold gives high confidence in the binding profile and allowed for consolidation of samples into 12×80-well master plates.

### Example 5—Rabbit B-Cell Culture Secondary Screening

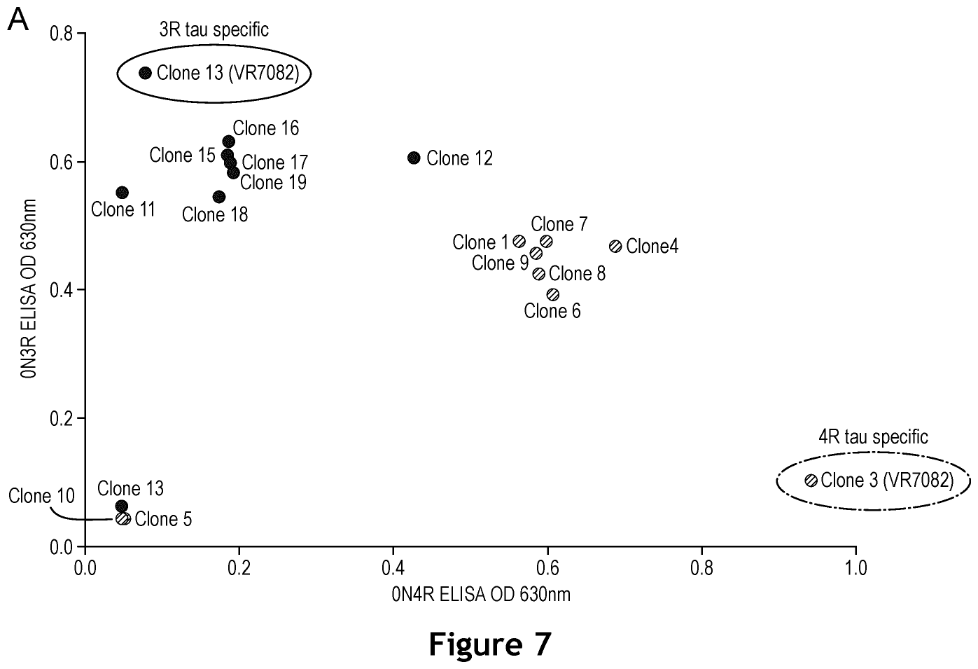

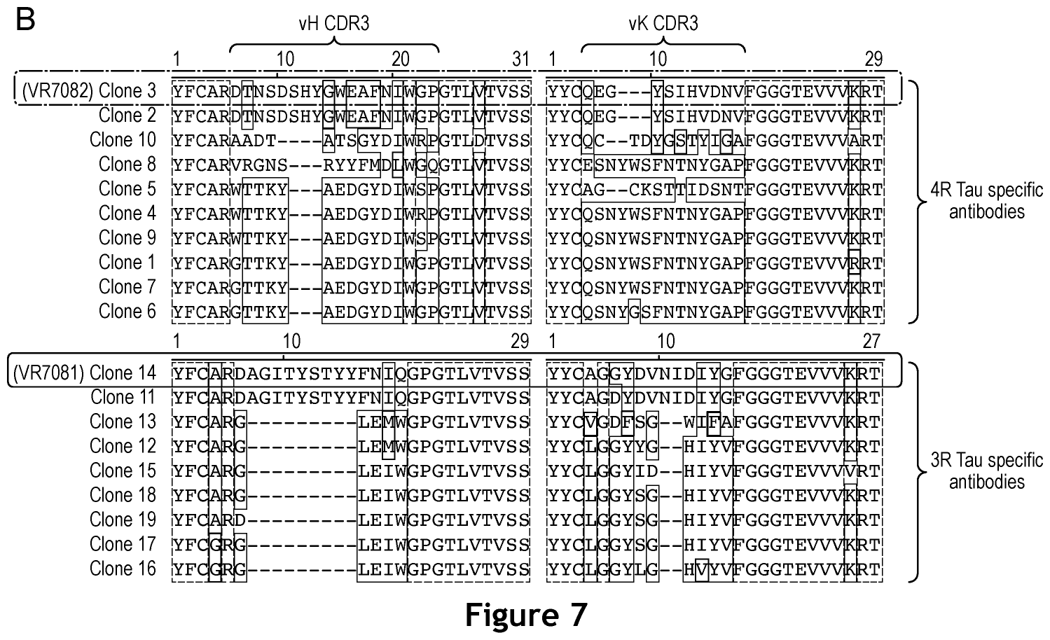

Following the identification of supernatants that showed reactivity to either TE10 or TE9/11 (FIG. 3) and cryopreservation of the activated B-cells, secondary supernatant screening was carried out to identify those wells that had antibodies demonstrating isoform specificity. This was achieved by ELISA using each of the peptide antigens (FIG. 4) and to recombinant 0N3 R and 0N4 R tau (FIG. 5)

Importantly, the remaining four tau isoforms are all identical within the MTBR and there are no other potential cross-reactive epitopes present within the rest of the protein.

The data obtained is shown in FIG. 4 shows the results of the peptide ELISA and FIG. 5 shows the results of the protein ELISA. All ELISA data is plotted as fold-change over background for each data point. Supernatants from wells which showed binding to single peptides and exhibited at least a 4-fold higher binding to the desired tau 0N isoform were selected for B-cell isolation and variable region gene recovery. In each case, the wells selected to be 3 R-specific are highlighted in red and the 4 R-specific wells are highlighted in green. It was interesting to note that some wells appeared to show cross-reactivity to both peptides. These are likely recognising a common epitope on the peptides. The lack of sequence homology between the two tau peptides suggests the cross reactivity was likely due to the linker that conjugates the peptide to the biotin molecule or carrier protein or polyreactive antibodies within the developing immune repertoire of the rabbits.

### Example 6—B-Cell Isolation, RT-PCR and Transcriptionally Active PCR Recombinant Transient Screening

B-cell isolation, via the fluorescent method, was performed on all of the selected wells highlighted red or green in FIG. 4 and FIG. 5. Reverse transcription and three rounds of PCR were carried out, to recover the variable region genes and create transfection ready linear expression cassettes. Table 3 shows the recovery levels from B-cell isolations, the number of PCRs and success of those PCRs from each well along with the specificity to either 3 R or 4 R tau for that well.

TABLE 3
B-cell isolation and PCR recoveries for selected wells

| | | | | | Kappa |
|---|---|---|---|---|---|
| | Tau | Number | Number | Heavy chain | chain |
| | isoform | of foci | PCRs | PCRs | PCRs |
| Well reference | specificity | picked | attempted | recovered | recovered |
| SSUP0011921834 | 4R | 8 | 5 | 5/5 | 5/5 |
| SSUP0011921958 | 4R | 8 | 5 | 4/5 | 5/5 |
| SSUP0011924815 | 4R | 0 | 0 | n/a | n/a |
| SSUP0011917532 | 4R | 4 | 4 | 4/4 | 4/4 |
| SSUP0011934401 | 4R | 8 | 5 | 5/5 | 5/5 |
| SSUP0011918493 | 4R | 8 | 5 | 5/5 | 5/5 |
| SSUP0011919075 | 4R | 7 | 5 | 5/5 | 5/5 |
| SSUP0011934305 | 4R | 8 | 5 | 5/5 | 5/5 |
| SSUP0011929468 | 4R | 8 | 5 | 4/5 | 4/5 |
| SSUP0011926345 | 4R | 8 | 5 | 5/5 | 5/5 |
| SSUP0011933382 | 4R | 8 | 5 | 5/5 | 5/5 |
| SSUP0011919104 | 4R | 8 | 5 | 5/5 | 5/5 |
| SSUP0011923507 | 3R | 0 | 0 | n/a | n/a |
| SSUP0011924325 | 3R | 0 | 0 | n/a | n/a |
| SSUP0011921782 | 3R | 0 | 0 | n/a | n/a |
| SSUP0011928511 | 3R | 8 | 4 | 4/4 | 4/4 |
| SSUP0011924609 | 3R | 8 | 4 | 4/4 | 4/4 |
| SSUP0011931102 | 3R | 8 | 4 | 4/4 | 4/4 |
| SSUP0011928754 | 3R | 8 | 4 | 3/4 | 4/4 |
| SSUP0011929699 | 3R | 8 | 4 | 4/4 | 4/4 |
| SSUP0011927754 | 3R | 8 | 4 | 4/4 | 4/4 |
| SSUP0011929686 | 3R | 8 | 4 | 4/4 | 4/4 |
| SSUP0011917961 | 3R | 8 | 4 | 4/4 | 4/4 |
| SSUP0011917987 | 3R | 8 | 4 | 4/4 | 4/4 |

In general, the results obtained demonstrated good B-cell isolation efficiencies, generating fluorescent foci from 83% of wells. Variable region recovery was observed from 97% of vH PCRs and 99% of vK PCRs. This represented an overall paired V-region recovery rate of 96%.

Linear expression cassettes, generated via the tertiary TAP PCR, were directly transfected into Expi293F cells. Following expression, the antibody containing supemnatants were assayed for JgG expression via ELISA. Expression of JIgG was observed in all cases where paired V-regions were recovered in the PCR. In total there were 77 samples containing JgG from 90 wells. All wells were subsequently assayed for binding to either 0N3 R or 0N4 R tau via ELISA. The ELISA data with one representative TAP expression per foci group is shown in FIG. 6. Wells selected for cloning and further testing are highlighted in green and red for the 4 R and 3 R specific antibodies respectively, in FIG. 6.

It is interesting to note that all except one of the foci wells picked retained their binding specificity to 3 R or 4 R tau following the TAP expressions. For the single antibody highlighted in black, that no longer appears to bind either 3 R or 4 R tau, it is likely that this was a false positive in the B-cell supernatant screening ELISA on protein (FIG. 5) and bound to a non-tau representative epitope on the TE10 peptide. This would explain how it was able to produce fluorescent foci, as these were performed using peptide, then subsequently not show binding to tau protein as a TAP transient.

### Example 7—Cloned Transient Screening and Sequencing of Rabbit IgGs

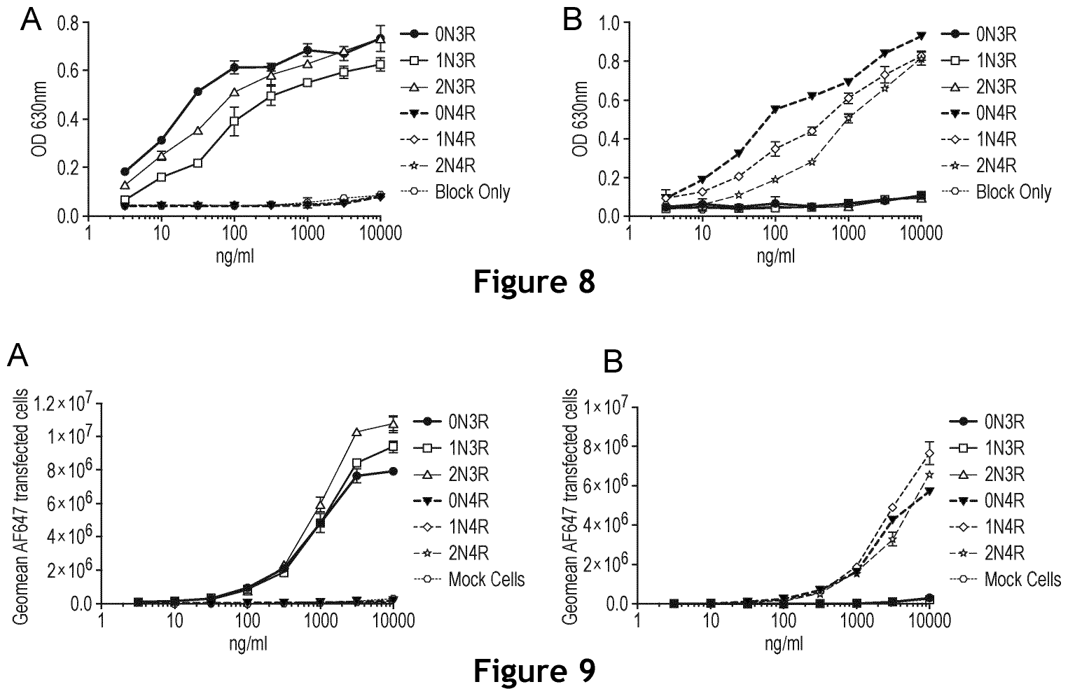

The variable region fragments, generated via PCR, were cloned as full-length rabbit IgGs. All cloned antibodies were sequenced, expressed as 30 ml transients in Expi293F cells and the resulting culture supernatants were assayed to determine the concentration of IgG they contained. All the supernatants were assayed via ELISA at 10 μg/ml IgG against 0N3 R and 0N4 R tau, to determine if they had retained their specificity profiles following cloning. FIG. 7A displays the ELISA data for all the cloned antibodies, whilst FIG. 7B shows the CDR3 sequences of these antibodies.

As can be seen, groups of antibodies with similar or the same antibody sequence clustered very closely together in their ELISA values, FIG. 7. There was a clear split between the 3 R and 4 R specific antibodies, with the 4 R specifics generally demonstrating more cross reactivity. Two antibody sequences, clone 3 (VR7082) (4 R-specific) and clone 14 (VR7081) (3 R-specific), that were considered to possess the suitable level of isoform-selectivity (without any measurable unwanted cross-reactivity) to justify larger scale expression/purification and more detailed characterisation. The sequences for the two antibodies and those of the subsequently generated scFv intrabodies generated are given in the Table below, which also includes other relevant sequences.

TABLE 4
Amino acid sequences including those of the VR7082 and VR7081 antibodies

| SEQ ID | |
|---|---|
| No. | Sequence |
| 1 | VR7082 - Light chain variable region |
| | AVVLTQTPAS VEAAVGGTVS INCQASEDIE SYLAWYQQKP GQRP |
| | KLLIYR ASTLASGVSSRFKGSGSGTE YTLTISGVEC ADAATYYCQ |
| | E GYSIHVDNVF GGGTEVVVK |
| 2 | VR7082 - FW1 - AVVLTQTPAS VEAAVGGTVS INC |
| 3 | VR7082 - CDR1 - QASEDIESYL A |
| 4 | VR7082 - FW2 - WYQQKPGQRP KLLIY |
| 5 | VR7082 - CDR2 - RASTLAS |
| 6 | VR7082 - FW3 - GVSSRFKGSG SGTEYTLTIS GVECADAATY YC |
| 7 | VR7082 - CDR3 - QEGYSIHVDN V |
| 8 | VR7082 - FW4 - FGGGTEVVVK |
| 9 | VR7082 - Heavy chain variable region |
| | QSVEESGGRL VTPGTPLTLT CTVSGIDLSN YAMIWVRQAP GEGLE |
| | YIGII SSTGNTFYAT WAKGRFTISK TSTTVDLKIT SPTAEDTATY F |
| | CARDTNSDS HYGWEAFNIW GPGTLVTVSS |
| 10 | VR7082- FW1 - QSVEESGGRL VTPGTPLTLT CTVS |
| 11 | VR7082 - CDR1 - GIDLSNYAMI |
| 12 | VR7082 - FW2 - WVRQAPGEGL EYIG |
| 13 | VR7082 - CDR2 - IISSTGNTFY ATWAKG |
| 14 | VR7082 - FW3 - RFTISKTSTT VDLKITSPTA EDTATYFCAR |
| 15 | VR7082 - CDR3 - DTNSDSHYGW EAFNI |
| 16 | VR7082 - FW4 - WGPGTLVTVS S |
| 17 | VR7082 - derived scFv intrabody |
| | MAQSVEESGGRLVTPGTPLTLTCTVSGIDLSNYAMIWVRQAPGEGL |
| | EYIGIISSTGNTFYATWAKGRFTISKTSTTVDLKITSPTAEDTATYFC |
| | ARDTNSDSHYGWEAFNIWGPGTLVTVSSGGGGSGGGGSGGGGSG |
| | GGGSAVVLTQTPASVEAAVGGTVSINCQASEDIESYLAWYQQKPG |
| | QRPKLLIYRASTLASGVSSRFKGSGSGTEYTLTISGVECADAATYYC |
| | QEGYSIHVDNVFGGGTEVVVK |
| 18 | VR7081 - Light chain variable region |
| | AQVLTQTPAS VSAAVGGTVS ISCQSSPSVL NTNDLSWFQQ KPGQ |
| | PPKVLI YGASTLASGV PSRFKGSGSG TQFTLTISDV QCDDAATYY |
| | C AGGYDVNIDI YGFGGGTEVV VK |
| 19 | VR7081 - FW1 - AQVLTQTPAS VSAAVGGTVS ISC |
| 20 | VR7081 - CDR1 - QSSPSVLNTN DLS |
| 21 | VR7081 - FW2 - WFQQKPGQPP KVLIY |
| 22 | VR7081 - CDR2 - GASTLAS |
| 23 | VR7081 - FW3 - VR7081 - GVPSRFKGSG SGTQFTLTIS |
| | DVQCDDAATY YC |
| 24 | VR7081 - CDR3 - AGGYDVNIDI YG |
| 25 | VR7081 - FW4 - FGGGTEVVVK |
| 26 | VR7081 - Heavy Chain variable region |
| | QSLEESGGRL VTPGTPLTLT CTVSGFSLSN YAMIWVRQAP GGGLE |
| | WIGTM GTNGRTYYAS WAKGRFTISK TSTTMDLSMT SLTAEDTAT |
| | Y FCARDAGITY STYYFNIWGP GTLVTVSS |
| 27 | VR7081 - FW1 - QSLEESGGRL VTPGTPLTLT CTVS |
| 28 | VR7081 - CDR1 - GFSLSNYAMI |
| 29 | VR7081 - FW2 - WVRQAPGGGL EWIG |
| 30 | VR7081 - CDR2 - TMGTNGRTYY ASWAKG |
| 31 | VR7081 - FW3 - RFTISKTSTT MDLSMTSLTA EDTATYFCAR |
| 32 | VR7081 - CDR3 - DAGITYSTYY FNI |
| 33 | VR7081 - FW4 - WGPGTLVTVS S |
| 34 | VR7081 derived scFv intrabody |
| | MAQSLEESGGRLVTPGTPLTLTCTVSGFSLSNYAMIWVRQAPGGGL |
| | EWIGTMGTNGRTYYASWAKGRFTISKTSTTMDLSMTSLTAEDTAT |
| | YFCARDAGITYSTYYFNIWGPGTLVTVSSGGGGSGGGGSGGGGSG |
| | GGGSAQVLTQTPAS VSAAVGGTVS ISCQSSPSVL NTNDLSWFQQ |
| | KPGQPPKVLI YGASTLASGV PSRFKGSGSG TQFTLTISDV |
| | QCDDAATYYC AGGYDVNIDI YGFGGGTEVV VK |
| 35 | Amino acid sequence of the 2N4R isoform of tau |
| | MAEPRQEFEVMEDHAGTYGLGDRKDQGGYTMHQDQEGDTDAGLKESPLQTPTE |
| | DGSEEPGSETSDAKSTPTAEDVTAPLVDEGAPGKQAAAQPHTEIPEGTTAEEAGIG |
| | DTPSLEDEAAGHVTQARMVSKSKDGTGSDDKKAKGADGKTKIATPRGAAPPGQ |
| | KGQANATRIPAKTPPAPKTPPSSGEPPKSGDRSGYSSPGSPGTPGSRSRTPSLPTPPT |
| | REPKKVAVVRTPPKSPSSAKSRLQTAPVPMPDLKNVKSKIGSTENLKHQPGGGKV |
| | QIINKKLDLSNVQSKCGSKDNIKHVPGGGSVQIVYKPVDLSKVTSKCGSLGNIHHK |
| | PGGGQVEVKSEKLDFKDRVQSKIGSLDNITHVPGGGNKKIETHKLTFRENAKAKT |
| | DHGAEIVYKSPVVSGDTSPRHLSNVSSTGSIDMVDSPQLATLADEVSASLAKQGL |
| 36 | TE9/11 peptide sequence - HQPGGGKVQIVYKP |
| 37 | TE10 peptide sequence - KDNIKHCPG |

### Example 8-3 R and 4 R Specific Antibody Characterisation

Following the identification of 3 R and 4 R tau specific antibodies, VR7081 and VR7082, their suitability for all further studies was assessed. That was done by characterisation in a range of assays against recombinant and natively expressed tau.

### (a) Recombinant Tau Binding and Characterisation Assays

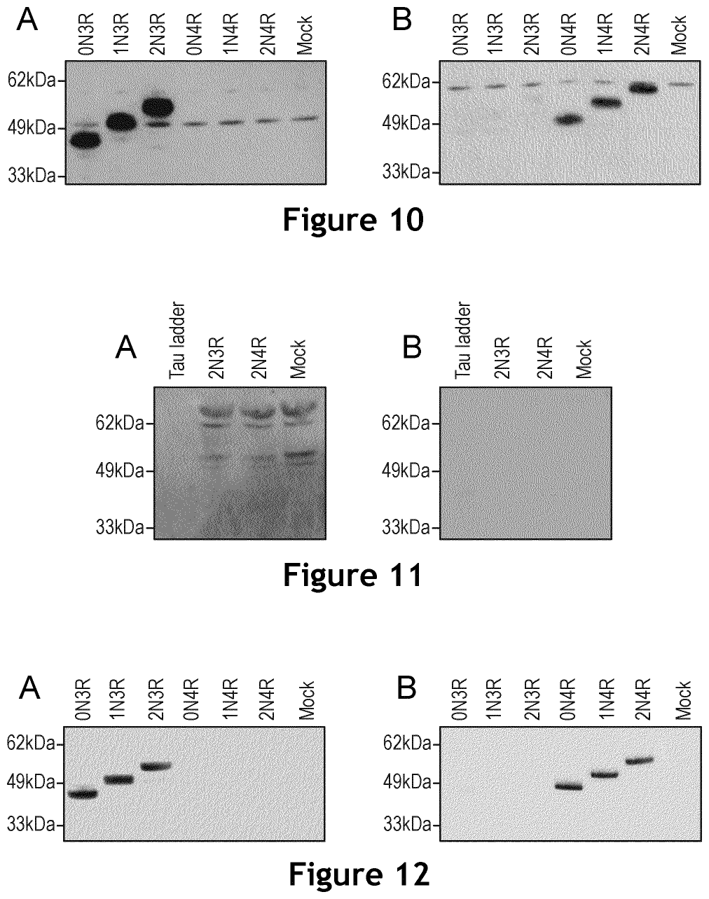

As described previously, VR7081 and VR7082 demonstrated exquisite specificity to 0N3 R and 0N4 R tau at 10 μg/ml via ELISA (FIG. 7). It was important to ensure this specificity had been retained following purification, therefore VR7081 and VR7082 were therefore tested against all six recombinantly expressed isoforms of tau via ELISA (FIGS. 8A and B).

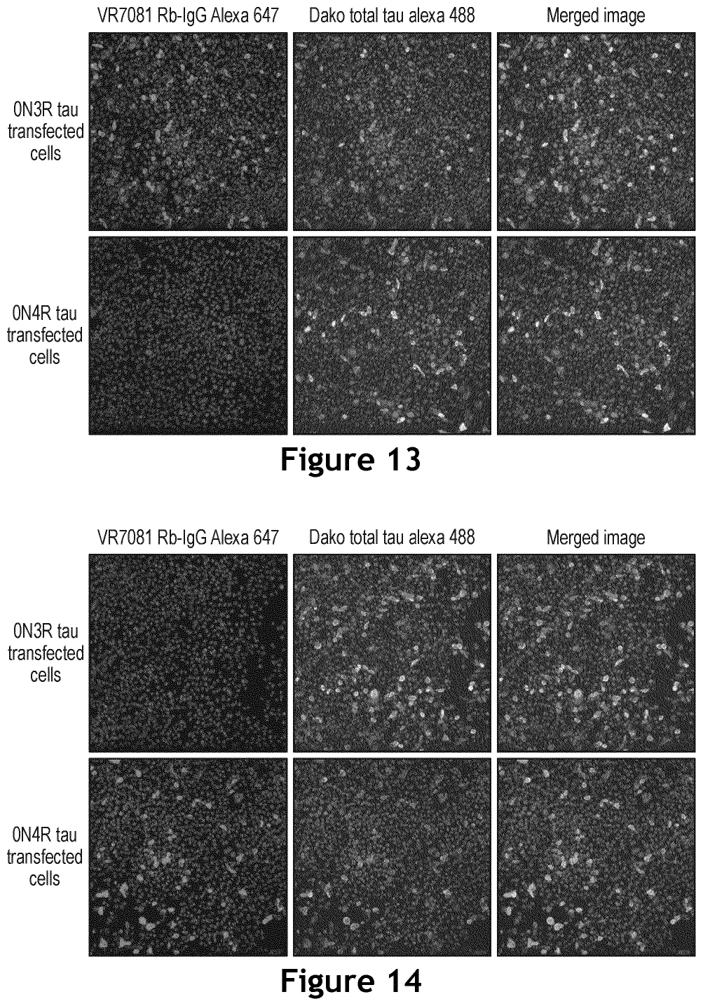

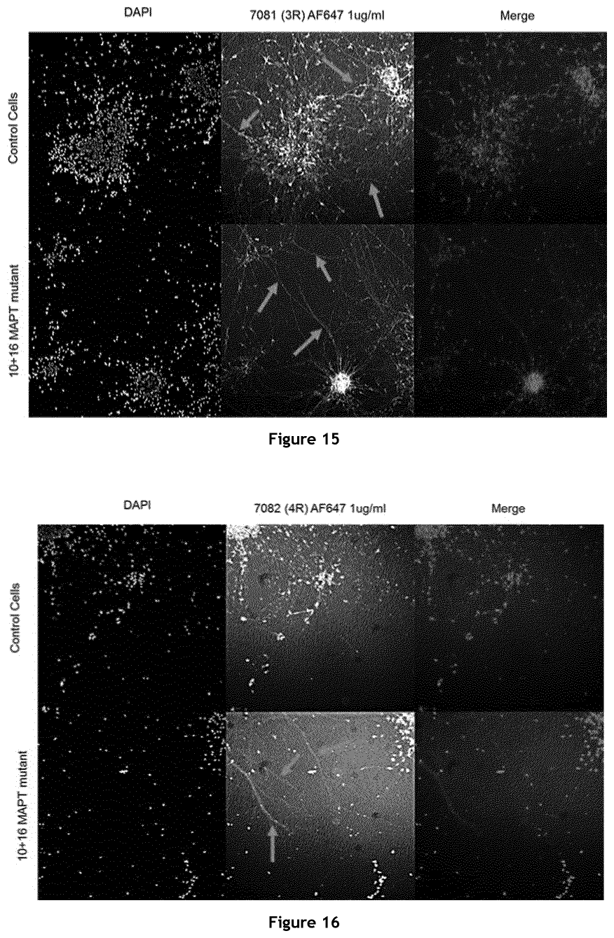

Following confirmation of the selectivity profiles of VR7081 and VR7082 to 3 R and 4 R via ELISA, FIG. 8, it was important to check the specificity of these antibodies against tau in a cellular context. The data for this flow cytometry assay can be seen in FIG. 9. FIG. 9 demonstrates that both VR7081 (A) and VR7082 (B) show absolute specificity to tau in a cellular context via flow cytometry. To ensure this specificity was retained in a Western blot, lysates were generated from cells overexpressing all the isoforms of tau and validated using the polyclonal anti-total tau antibody. The Western blots with VR7081 and VR7082 can be seen in FIGS. 10 A and B respectively. Peptide TE9/11 and peptide TE10 represent linear epitopes within across the tau exon 9/11 boundary, unique to 3 R tau, and within exon 10, unique to 4 R tau. These epitopes were therefore unlikely to be affected via the sample reduction performed for Western blot. FIG. 10 shows that both VR7081, FIG. 10 A and VR7082, FIG. 10 B retain their specificity profiles via Western blot to 3 R and 4 R tau respectively.

Both antibodies also appeared to show reactivity with a non-tau band of approximately 49 kDa (VR7081) or 60 kDa (VR7082). It is unclear what is driving this extra band, it could be a result of the Rabbit IgGs, the secondary HRP conjugated antibody or driven through off target variable region binding.

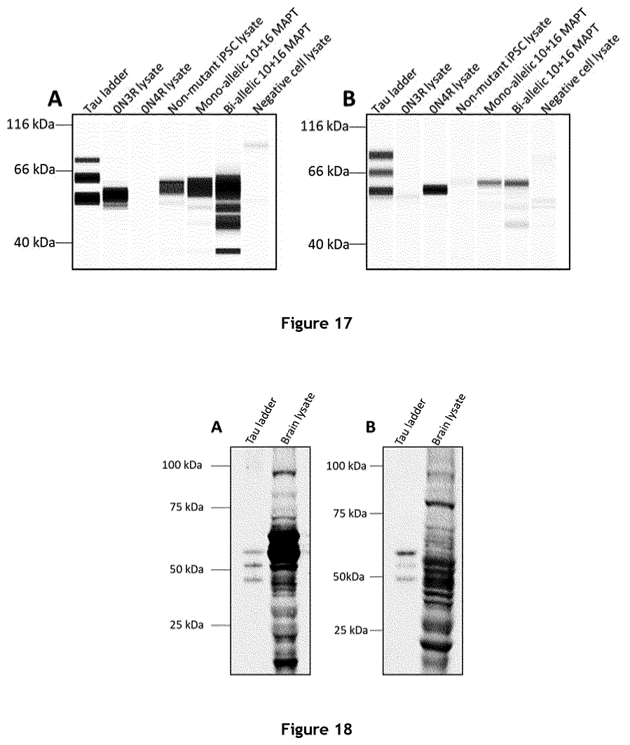

To address this issue, a Western blot was performed with 2N3 R and 2N4 R tau containing lysate as well as the recombinant tau ladder and mock lysate and probed with a non-tau reactive rabbit or mouse IgG with the results shown in FIG. 11. The Western blots in FIG. 11 A show a similar nonspecific banding pattern to VR7081 and VR7082 with non-specific bands around 60 kDa and 42 kDa with the non-tau reactive rabbit IgG. However, in FIG. 11 B, with non-tau reactive mouse IgG these bands are not observed. The results clearly indicate that any non-tau bands observed in FIG. 10 are because of what the rabbit IgG constant regions or HRP reveal rather than the tau specific variable regions.

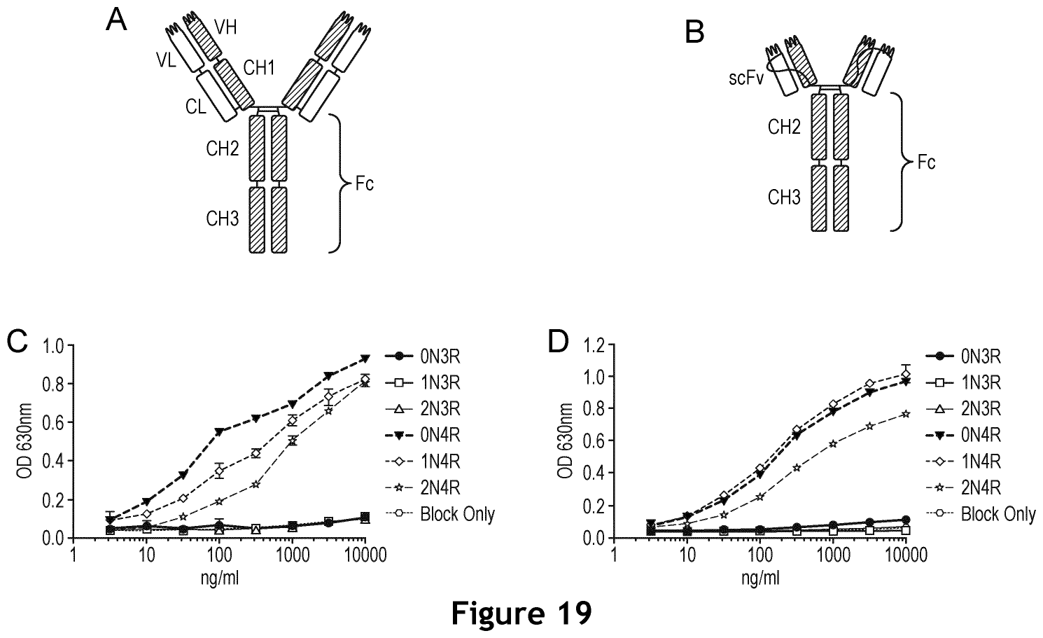

Further experiments were then performed to ensure that the antibodies were of more general utility. To ensure this was possible the rabbit IgGs were converted to mouse IgGs with rabbit variable regions. This was performed as the non-tau reactive mouse IgG showed no banding in tau over expression lysates, FIG. 11 B. The resulting chimeric antibodies were then assayed via Western blot against individual tau isoform overexpressing cell lysates. The results of these blots can be seen in FIG. 12. It is clear to see, from FIG. 12, that the conversion to a mouse chimeric antibody with both VR7081 and VR7082 has removed the non-tau band from the blots. This data, along with the banding seen with a non-tau antibody in FIG. 11 A, demonstrates that this non-tau reactive band was a result of the rabbit IgG constant regions or the secondary HRP conjugated antibody used for Western blotting. It is important to note that this is a Western blot artefact, likely due to the presentation of linear epitopes of other cellular proteins, as this non-tau reactivity is not observed in the same cells in a flow cytometry assay.

The conversion to a mouse IgG proved very successful in helping the specificity of these antibodies via Western blot, however as discussed previously this non-tau reactive band is not a problem for the experiments performed so, unless otherwise stated all data is generated with the rabbit IgG versions of these antibodies.

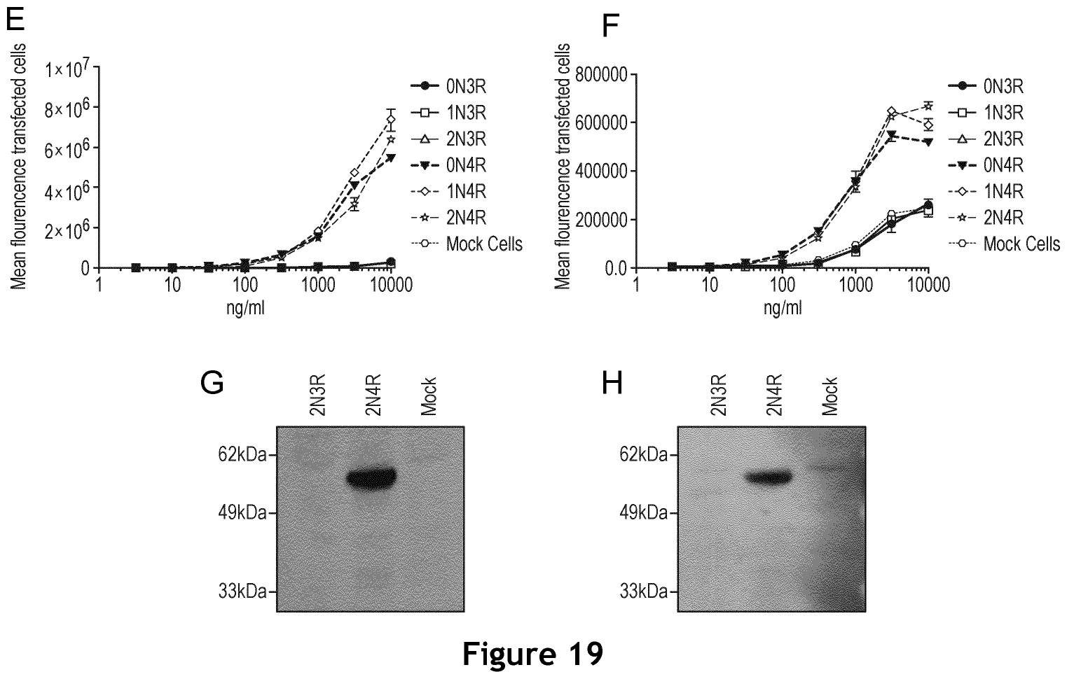

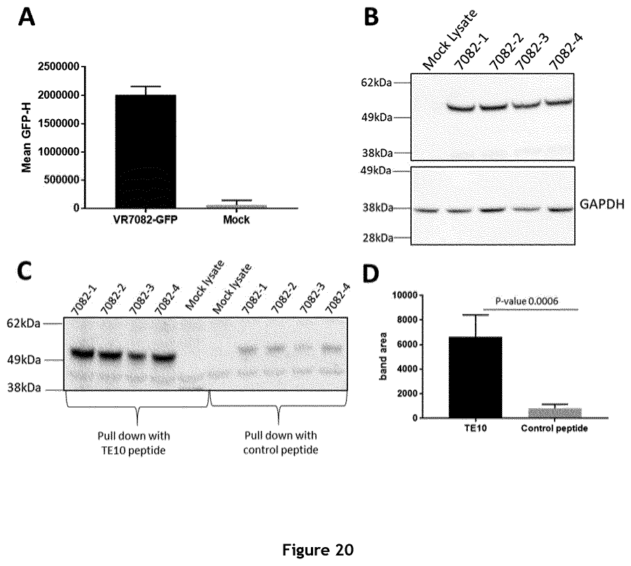

Another key area it was important to assess of these antibodies in was immunofluorescence. To assess these antibodies 0N3 R and 0N4 R tau was overexpressed in adherent CHOK1 cell before being fixed, permeabilised and co-stained with either VR7081/VR7082 and the polyclonal anti-total tau antibody. The resulting immunofluorescent data can be seen in FIG. 13 for VR7081 and FIG. 14 for VR7082. In each case with VR7081 and VR7082 staining with that antibody was only seen for cells expressing the tau protein that the antibody is specific for. The data shown in FIG. 13 and FIG. 14 clearly demonstrates both VR7081 and VR7082 retain exquisite specificity for 3 R and 4 R tau respectively via immunofluorescence. Polyclonal anti-total tau antibody staining enables visualisation of all the cells expressing tau within these populations. It is therefore important to note that in both cases the staining with either VR7081 or VR7082 overlay with all polyclonal anti-total tau antibody staining for the appropriate tau isoform.

The data obtained therefore clearly shows that VR7081 and VR7082 are highly specific for 3 R and 4 R tau respectively. In all assays that were performed, both antibodies showed no off-target binding, to the non-desired tau isoforms.

### (b) Native Tau Binding and Characterisation Assays

Binding to tau expressed in a native system of iPSC derived neurones or from human brain samples was next demonstrated.

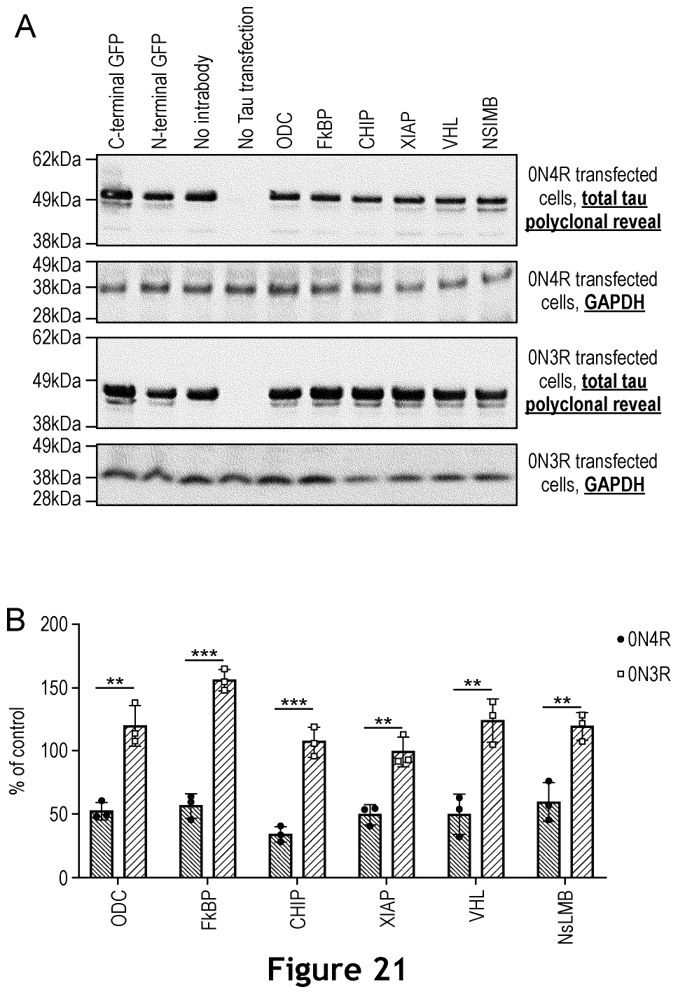

Binding of VR7081 and VR7082 was initially assed via immunofluorescence on iPSC-derived neurons. Both control neurons with no tau mutation (express only 0N3 R tau) or mono allelic 10+16 MAPT mutant neurons (that express both 0N3 R tau and 0N4 R tau) were used. The resulting images can be seen in FIG. 15 for VR7081 and FIG. 16 for VR7082.

As expected VR7081 binds to 3 R tau in both the non-mutant control cells and in the 10+16 MAPT mutant neurones, FIG. 15. VR7082 demonstrates binding to 4 R tau within the 10+16 mutant neurones, FIG. 16, and as expected shows no binding to the non-mutant control neurones, as these express no 4 R tau.

Specificity was also ensured in immunoblot using lysates from control, 10+16 monoallelic and biallelic MAPT mutation neurons via Peggy Sue Simple Western together with lysates from ExpHEK-293F cells over expressing 0N3 R or 0N4 R tau as well as tau negative cell lysates and a recombinant tau ladder containing all tau isoforms (FIG. 17). It can be seen from the blots in FIGS. 17A and B that both VR7081 and VR7082 retain their specificity in the over expression lysates to both 3 R and 4 R respectively. VR7081 can be seen to bind to a 0N3 R tau sized band in all three of the iPSC lysates as expected. As previously discussed at this age the non-mutant neurones do not expresses 4 R tau. Therefore, as expected, VR7082 demonstrates binding to 0N4 R tau sized band in only the 10+16 mutant iPSC derived neurones.

It is interesting to note with both VR7081 and VR7082 the non-tau bands observed in FIG. 10 are no longer seen via this method. This blot uses a different secondary antibody to a standard Western blot and it appears that this has solved the non-tau banding. This gives a further indication that this non-tau band observed with both VR7081/VR7082 and the non-tau rabbit antibody, FIG. 10 and FIG. 11, were a result of the secondary HRP conjugated antibody.

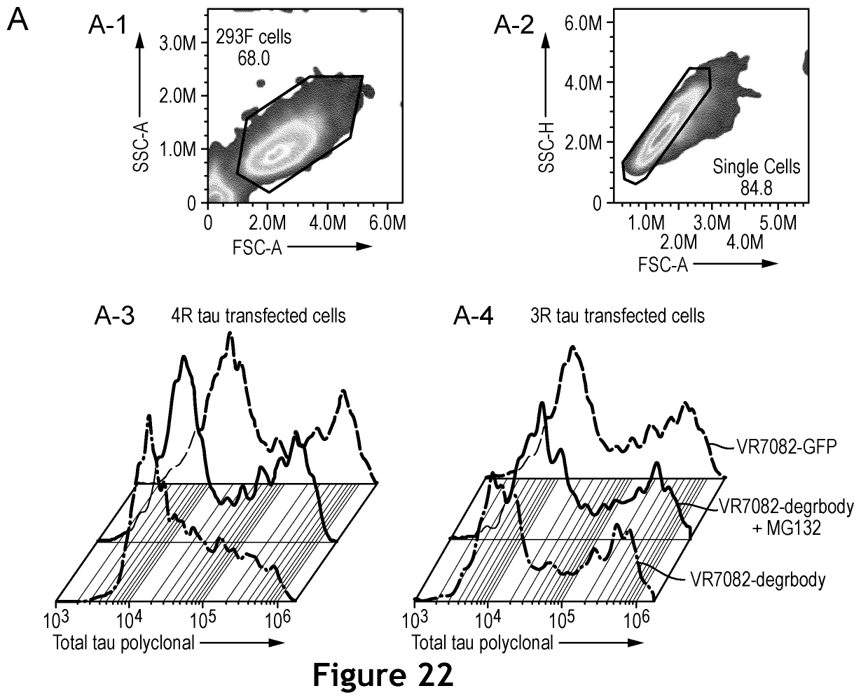

Tau is able to undergo significant post translational modification (Ercan et al., 2017, supra; Kontaxi et al., 2017, supra; Mair et al., 2016, supra). It was therefore important to confirm that both VR7081 and VR7082 were able to bind 3 R and 4 R tau, respectively, regardless of the post translational modification state of tau. Aged brain lysate contains a range of tau species with many post translational modifications and tau truncations. It therefore, represented a good source of material to test in Western blot and confirm that both VR7081 and VR7082 were able to bind to many forms of tau (FIG. 18).

Multiple bands are observed in both blots with either VR7081 or VR7082 FIG. 18, importantly however three distinct and different bands are present in the tau ladder for VR7081 and VR7082. The multiple bandings with the brain lysate are likely due to multiple phosphorylation states of tau, higher molecular weight aggregates of tau and or truncated tau variants. This data is consistent with other data using these lysates. This data gives a high degree of confidence that both VR7081 and VR7083 are capable of binding to all post translational modification states of 3 R and 4 R tau.

The data shown in the section on “Native tau binding and characterisation assays” demonstrates that VR7081 and VR7082 are both able to bind to natively expressed tau in a range of assays. It has also been demonstrated in FIG. 17 that the non-tau banding observed in standard Western blot was due to the secondary HRP conjugated antibody. Finally, FIG. 18 demonstrates that both VR7081 and VR7082 are capable of binding tau in a range of post translational modification states.

### Example 9—Generation of 4 R Degrading Tau Degrabody

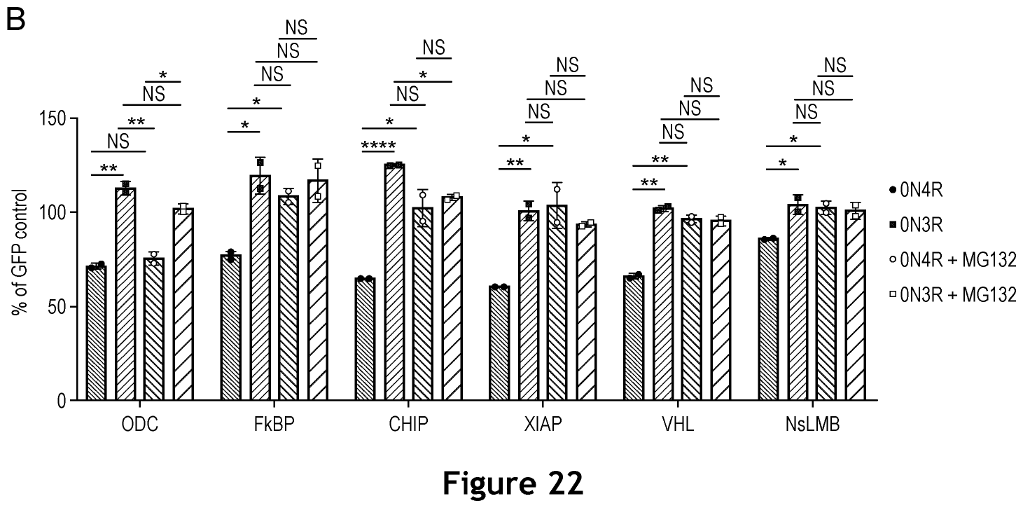

IgGs or derivatives of such as Fab fragments, cannot be used as intracellular antibodies as their disulphide bonds do not form correctly in the reducing environment of the cytoplasm. This ensures that the heavy and light chains do not associate and hence no binding moiety is formed. However, IgGs can be re-formatted to non-disulphide stabilised scFvs allowing for use as intrabodies. In the scFv format the heavy and light chain variable regions are physically linked by a flexible linker. This ensures the heavy and light chains pair correctly inside the cell and form a binding moiety. To ensure that VR7082 reformatted in that way retained its binding properties, the IgG was initially re-formatted into an scFv-msFc. This is a screening construct where the scFv is fused to a mouse Fc to allow for detection with anti-mouse secondary antibodies, a diagrammatic representation of IgG and scFv-Fc can be seen in FIG. 19A and FIG. 19B respectively. VR7082 scFv-Ms-Fc was tested. The results of the VR7082 scFv-Ms-Fc conversions can be seen in FIG. 19. The conversion was tested via ELISA (FIG. 19D), flow cytometry (FIG. 19F) and Western blot (FIG. 19H). For comparative purposes the data seen in FIG. 19C and FIG. 19E is ELISA and flow cytometry data respectively. FIG. 19G is Western blot data utilising VR7082 IgG for comparison with the scFv format of VR7082.

To allow for intracellular cytoplasmic expression of an antibody fragment, or any other protein, expression vectors are designed devoid of any specific leader sequence. Tau exists predominantly within the cytoplasm of the cell, so for the intrabody work it was important that the intrabodies were also expressed in the cytoplasm. Expression constructs were therefore designed without any leader or localisation signals. Despite the successful conversion of VR7082 to an scFv format (FIG. 19), it was important to ensure that the scFv fragment was capable of cytoplasmic expression and specific binding to the immunising peptide. To allow for testing of VR7082 as a scFv intrabody a fusion construct with GFP was designed, by fusing GFP to VR7082-scFv with a flexible linker. GFP was chosen as it could be used as a surrogate for future fusions and it allows for detection of expression in live cells, via fluorescence, and detection of intrabody via Western blot, via anti-GFP antibodies.

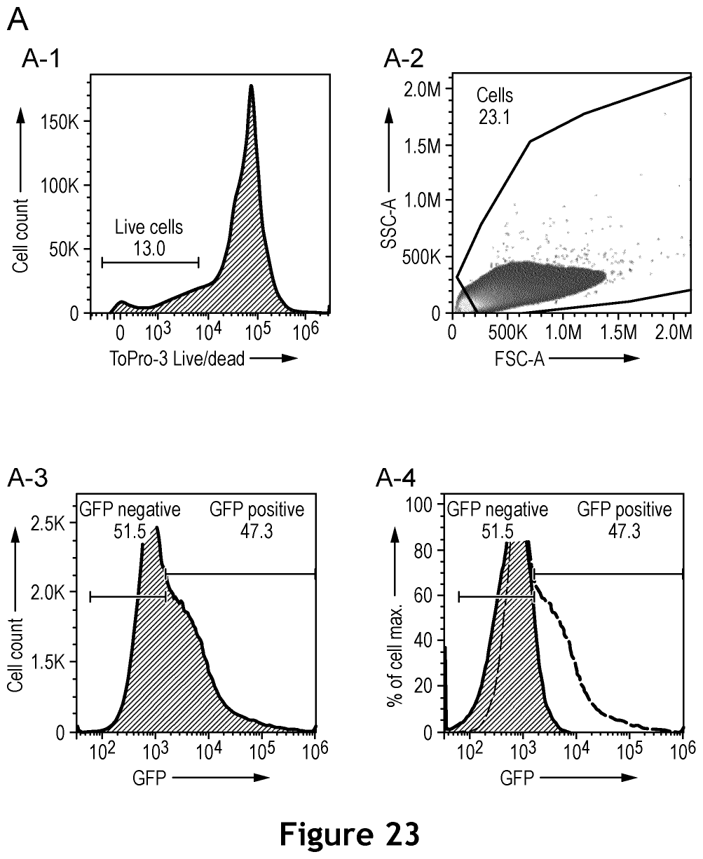

The subsequent expression construct was transfected (n=4) into HEK-293F cells. These were then assessed for GFP fluorescence via flow cytometry (FIG. 20A) and the cells were lysed. The lysates were then assayed via Western blot (FIG. 20B), to ensure the intrabody GFP fusion was intact and the correct molecular weight. Finally, these lysates were used in a pull-down experiment where TE10, the 4 R tau specific peptide, was used to pull down the intrabody before subsequent Western blot this was compared to a pull down with an irrelevant mix of peptide controls (FIG. 20C and FIG. 20D).

It can be seen from this data that the intrabody-GFP fusion was expressed in HEK-293F cells, and the GFP folded correctly as indicated by GFP fluorescence (FIG. 20A). To demonstrate that VR7082 is correctly fused to GFP a Western blot of transfected HEK-293F cells with a GFP antibody was performed. If intact, scFv-GFP fusion intrabody should be observed at a molecular weight of approx. 53 kDa (scFv 26 kDa+linker 1 kDa+GFP 26 kDa). Western blotting of the cell lysates (FIG. 20B) indicates that this construct was expressing intact fusion protein, with a single band present at 53 kDa. Finally, the intrabody was assessed for binding to its immunising peptide. The Western blot (FIG. 20C) shows that the where the VR7082-scFv-GFP lysates were pulled down with TE10 coated beads (lanes 1-4) a band the size of VR7082-scFv-GFP is observed. However, when the VR7082-scFv-GFP lysates are pulled down with irrelevant peptide coated beads (lanes 7-10) or when mock transfected lysate is pulled down with TE10 coated beads (lane 5-6) no band is observed (FIG. 20C). Band densitometry from this blot, (FIG. 20D), indicated that the scFv-GFP intrabody construct is still capable of specifically binding the TE10 peptide.

This phase of work showed the successful conversion of VR7082, a 4 R tau specific rabbit IgG, to an scFv-Ms-Fc and an scFv-GFP intrabody both of which still demonstrated specific binding to 4 R tau or 4 R tau peptide. The next phase of work was to focus on the conversion ofthis intrabody into a target degrading intrabody, namely a degrabody.

### Example 10—Generation of a degrabody from VR7082-scFv intrabody

To convert VR7082-scFv intrabody into a degrabody capable of degrading 4 R tau, the GFP domain was replaced with one ofsix degradation domains that were selected from the literature. These domains were fused as an N or C terminal fusion to VR7082-scFv, dependent on the orientation suggested by the literature (Table 5).

TABLE 5
Degradation domains selected for
fusion to VR7082-scFv intrabody to form a degrabody.

| | | Nor C | | |
|---|---|---|---|---|
| | | terminal | | |
| Degradation | | fusion to | Method of | Literature |
| domain | Acronym | scFv | degradation | reference |
| C-terminal sequence | ODC | C terminal | Ubiquitin | Zhao, Pferdehirt, |
| of Ornithine | | | proteasome | and Segatori 2018 |
| decarboxylase | | | independent | |
| Human FκBP-12 | FκBP | C terminal | Ubiquitin | Banaszynski et al. |
| protein | | | proteasome | 2006 |
| | | | independent | |
| C-terminus of | CHIP | C terminal | Ubiquitin | Portnoff et al. 2014 |
| Hsc70 interacting | | | proteasome | |
| protein | | | independent | |
| X-linked inhibitor | XIAP | C terminal | Ubiquitin | Gross et al. 2016 |
| of apoptosis protein | | | proteasome | |
| | | | independent | |
| Von Hippel-Lindau | VHL | N terminal | Ubiquitin | Fulcher et al. 2017 |
| protein | | | proteasome | |
| | | | independent | |
| N-terminus of Slmb | NS1mb | N terminal | Ubiquitin | Caussinus, Kanca, |
| protein | | | proteasome | and Affolter 2011 |
| | | | independent | |

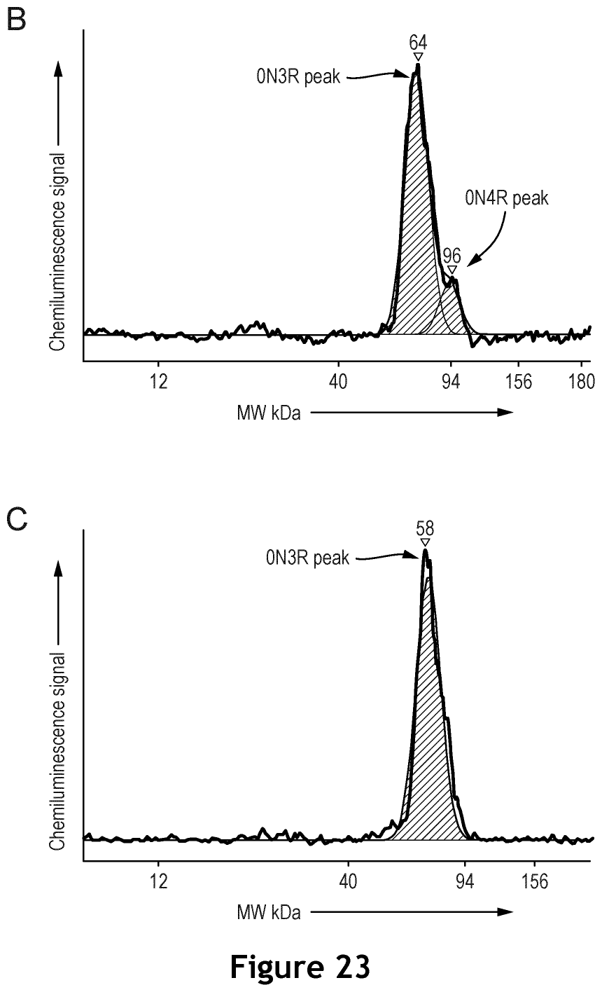

All degrabody fusion constructs as well as GFP N and C terminal fusions (as non-degrading intrabody controls), were co-transfected into ITEK-293F cells with either 0N3 R or 0N4 R tau. 48 h post-transfection, the cells were lysed and analysed via Western blot utilising the polyclonal total tau antibody (Dako). Band densitometry was then carried out and values were expressed as a percentage of the GFP fusion control. A representative Western blot (FIG. 21A) and the data for the n=3 densitometry from the tau Western Blots (FIG. 21B) can be seen.

To allow for accurate comparison of degradation each construct is expressed as % tau compared with the non-degrading VR7082-GFP control (fused to the respective N or C termini) (FIG. 21B). This was important as it shows the effect of the degrabody over and above a binding, but non-degrading intrabody.

The data presented in FIG. 21 demonstrates that the co-transfection of VR7082 degrabody with 0N4 R tau induces the degradation of 0N4 R compared with the non-degrading VR7082-GFP intrabody control. It is also possible to see that this is not the case when the degrabody constructs are co-transfected with 0N3 R tau.

Following this work, it was interesting to determine if the presence of MG132 (a proteasome inhibitor) would stop the degradation of 4 R tau. This should be the case in all degrabodies except for ODC, which has been previously demonstrated to be less dependent on the ubiquitin proteasome (Erales and Coffino, 2014, Biochim Biophys Acta, 1843 (1), 216-21). Degrabody constructs were co-transfected with 0N4 R or 0N3 R tau in HEK-293F cells, following a 6-hour incubation, MG132 was added to half of the cultures. All cultures were then incubated for a further 18 hours before analysis via flow. Flow cytometry was used rather than western blotting in these experiments as it allowed for higher throughput screening of all the degrabody constructs against 0N3 R and 0N4 R tau with and without MG132 treatment. Whilst technically possible by Western Blotting the numbers of blots required meant this technique was much less practical when testing many conditions and constructs. It was also important to ensure that any degradation was not a Western blotting artefact and that it could be confirmed by multiple techniques. Representative flow cytometry plots and comparative assay data can be seen in FIG. 22A and the geomean of all single cell populations co-transfected with VR7082 degrabodies normalised to VR7082-GFP levels can be seen in FIG. 22B.

The gating strategy for this assay was to first enable identification of HEK-293F cells (FIG. 22A-1) followed by single cell identification via forward scatter area compared with height (FIG. 22A-2). The single cells were then assessed for intracellular tau staining via polyclonal total tau antibody (Dako) in either 0N4 R—(FIG. 22A-3) or 0N3 R— (FIG. 22A-4) transfected cells.

As described above for FIG. 21B in FIG. 22B, all tau levels are expressed as a % of the VR7082-GFP (binding but non-degrading intrabody) control. The data in FIG. 22 clearly demonstrates in a separate assay system that all of the degrabody constructs are capable of degrading 0N4 R tau whilst retaining levels of 0N3 R tau within HEK-293F cells. With a statistically significant reduction of 4 R tau levels compared to 3 R tau levels (via T-test) for all constructs (FIG. 22 B). All fusions except for ODC were proteasome-dependent as MG132 treatment blocked the degradation of 4 R tau (FIG. 22 B), With a statistically significant difference (via T-test) for all 0N4 R co-transfections with an without the addition of MG132 (FIG. 22 B). Once again this is not the case with 0N3 R tau transfected cells which show no statistical difference between the MG132 treated and untreated groups (FIG. 22 B) indicating that treatment with MG132 is effecting only the proteasome degradation and not the general tau proteostasis within the cell.

The data presented in FIG. 21 and FIG. 22 clearly demonstrate that VR7082-scFv intrabody has been successfully converted into an scFv degrabody capable of the specific degradation of 0N4 R tau.

### Example 11—Degradation of 4 R Tau Using VR7082 Degrabody in iPSC Derived neurones

In transfection of neurons via lipid-based transfection methodologies only a small percentage of cells receive transfected DNA (Karra and Dahm 2010). For this reason, viral transfection using Adeno-associated virus (AAV) was selected as the method of delivery for degrabodies and controls for this project. Three AAV constructs were acquired from GeneCopeia, two of which contained VR7082-scFv fused to either the XIAP degradation domain or Halo tag (non-degrading control) as well as an AAV vector negative control with a construct lacking an. The VR7082-XIAP construct was also acquired with and without IRES expression of a GFP transfection marker.

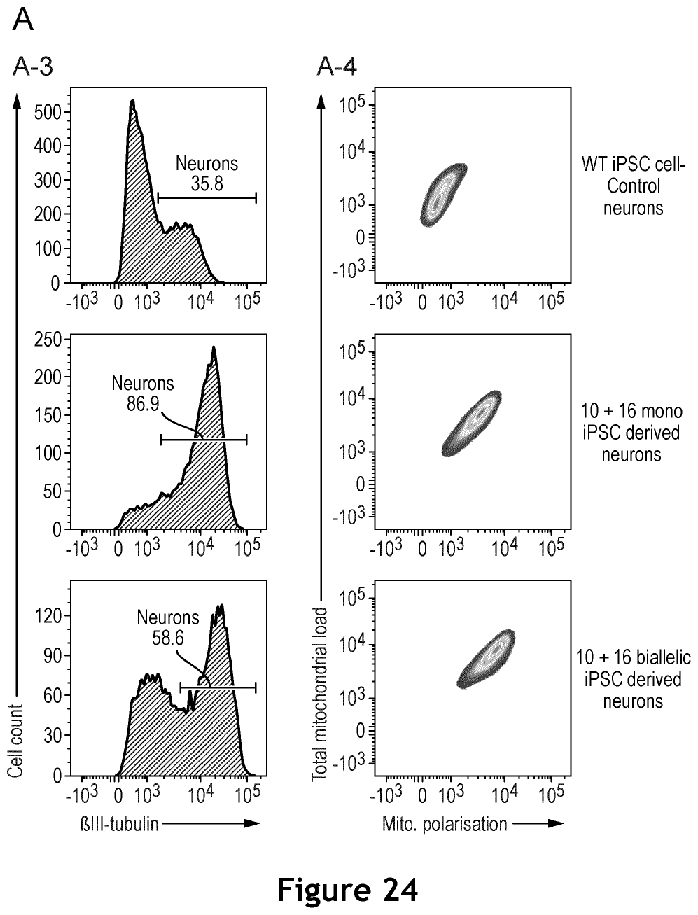

The VR7082-XIAP+IRES GFP AAV was initially used to transfect the 10+16 biallelic MAPT mutant, neurons at day 90 post induction. Cells were analysed seven days post-transfection to allow for expression of the degrabody and GFP transfection marker. Two populations of cells were then identified and sorted using flow cytometry based on GFP positive or negative signal (FIG. 23A-3). These two populations were lysed and tau protein levels were assessed using Peggy Sue simple Western (FIG. 23B and FIG. 23C). The full gating strategy can be seen in FIG. 23A.

Live cells were initially identified via lack of ToPro3 staining (FIG. 23A-1). Intact cells were identified via FCS-A v SSC-A (FIG. 23A-1-2). It is interesting to note that there are lots of events that are ToPro3 negative but are very small and in the debris area (bottom left of the plot) of the FSC-A v SSC-A plot. It is possible they are cellular debris that has formed a small lipid micelle. Such debris would in theory have an intact lipid bilayer membrane and as such would exclude the ToPro3 stain (in the way a live cell does). Finally, cells were sorted two ways based on GFP expression (FIG. 23A-3).

Following sorting, the two cell populations were lysed and then run on a Peggy Sue Simple Western. The resulting traces can be seen from the sorted GFP-negative cells (FIG. 23B) and the sorted GFP-positive cells (FIG. 23C). The trace for the GFP-negative cells displays two peaks, a large peak representative of 0N3 R, and a small peak corresponding to 0N4 R tau (FIG. 23B). In contrast, the GFP-positive cells had a peak representing 0N3 R tau, but no peak representing 0N4 R tau was detected (FIG. 23B). This suggests that 0N4 R has been degraded in cells transduced with XIAP.

Taken together, the data presented in FIG. 23 demonstrates that it is possible to deliver a degrabody to iPSC derived neurons and degrade 4 R tau protein from these cells.

### Example 12—Phenotypic Effect of 4 R Tau Removal on iPSC Derived Neurons

Previous work has shown that GFP can be toxic to neuronal cells (Detrait et al. 2002, Mol Ther, 5 (6), 723-30]. As the effect of GFP on the mitochondrial polarisation assay is unknown, for this work an AAV vector was used without GFP. For the same reason a VR7082-Halo tag intrabody as a non-degrading control rather than the previously used VR7082-GFP intrabody.

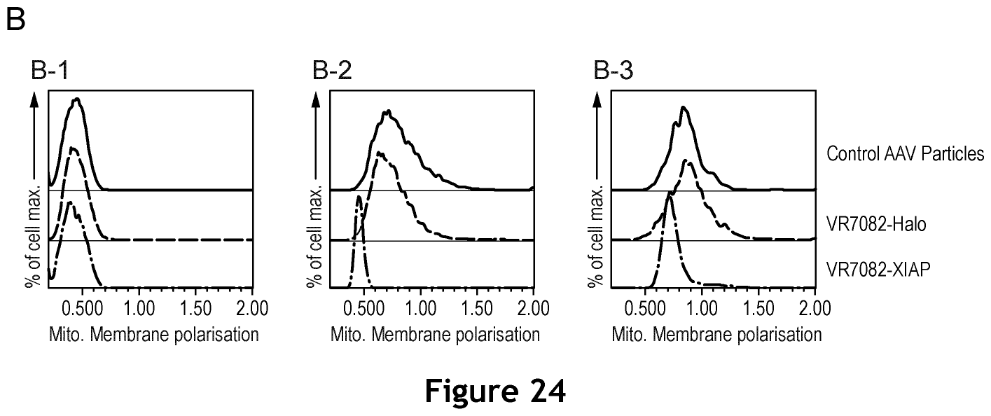

Neurons were differentiated from WT, 10+16 mono and 10+16 biallelic MAPT mutant iPSC lines cell lines. At day 80-90 the lines were treated with AAV containing VR7082-XIAP, VR7082-Halo or a negative AAV construct and were incubated for a further 7 days. Following incubation the cells were run in a mitochondrial membrane polarisation assay. In which mitochondrial membrane polarisation is assessed via staining with MitoTracker Deep Red CMX ROS and normalised to total mitochondrial load via staining with Mito Tracker deep red. The resultant data can be seen in FIG. 24.

The gating strategy for this assay is to identify intact neuronal cells which can be assayed for mitochondrial membrane polarisation and total mitochondrial load (FIG. 24A). A representative example of each treatment group on each iPSC derived neuronal cell line (FIG. 24B) demonstrates that in WT control neurons, (FIG. 24B-1) there is no effect of either the control AAV, VR7082-Halo or VR7082-XIAP treatment groups. However, when these treatments are applied to 10+16 mono neurons, (FIG. 24B-2) or 10+16 biallelic neurons, (FIG. 24B-3) treatment with VR7082-XIAP degrabody reduced the level of mitochondrial membrane polarisation. This was observed in n=5 neuronal inductions for each genotype and treatment group (FIG. 24C). As expected, no statistically significant difference in mitochondrial polarisation was observed in 10+16 MAPT mutant neurons between the control AAV particle treatment and VR7082-Halo intrabody for the 10+16 mono or 10+16 biallelic lines (p=0.145+p=0.922, ANOVA). This is because although the VR7082-Halo intrabody will bind to 4 R tau within these cells, it will not induce degradation of 4 R tau. However, when cells were transfected with VR7082-XIAP degrabody a statistically significant decrease of mitochondrial polarisation is observed in both the 10+16 mono and 10+16 biallelic mutant cell lines from the control AAV (p=<0.0001+p=0.0052, ANOVA) and from the VR7082-Halo treated groups (p=<0.0001+p=0.0018, ANOVA) (FIG. 24C). It is interesting to note that whilst only a modest decrease of the mutant phenotype is observed in the 10+16 biallelic neurons, in the 10+16 mono neurons the level of mitochondrial polarisation is reduced to levels that are not significantly different from the WT neurons (p=0.567, ANOVA) (FIG. 24C).

Because the VR7082-RbIgG 4 R specific antibody has the same epitope as the VR7082-scFv degrabody, it was not possible to assess 4 R tau levels in these experiments. However, it has been previously demonstrated (FIG. 21, FIG. 22 and FIG. 23) that VR7082-XIAP specifically induced 4 R tau degradation and not 3 R degradation. It was confirmed that there was no change in 3 R tau levels between any of the treatment groups, suggesting no off target degradation effect in these experiments (FIG. 24D).

Here have we successfully demonstrated that the VR7082 degrabody can be applied to 10+16 MAPT mutant iPSC-derived neurons to restore non-mutant levels of mitochondrial membrane polarisation.

### Example 13—Epitope Characterisation of VR7082 Antibody

Intracellular tau expression constructs were designed to make single alanine point mutations across the peptide epitope TE10 as well as the P301S and L mutations common in disease models of tauopathy. The constructs were synthesised by TWIST bioscience into a vector suitable for the intracellular expression of tau.

Upon receipt of the constructs, single constructs were transiently transfected into Expi293 cells (Thermo-Fisher) according to the instructions from the manufacturer. The cells were incubated at 38° C., shaking at 220 RPM, in a humidified 5% CO2 environment. Following a 48 hour incubation, the cells were harvested via centrifugation at 350 G(av) for 10 minutes. The cells were fixed and permeabilized with Invitrogens Fix and Perm kit, according to the instructions from the manufacturers. The cells were then stained with a non-VR7082 cross blocking antibody HT7 labelled with alexa-488, for 30 minutes on ice before washing in PBS+1% BSA, and then seeded at 20,000 cells/well. Each well was subsequently stained with 10 μg/ml VR7082 for 30 minutes on ice and the cells were washed once in PBS+1% BSA and a secondary anti-rabbit-Fc-Alexa647 (Jackson Laboratories) reveal antibody was added at 1:1000 dilution. The cells were incubated on ice for 30 minutes before being washed in PBS+1% BSA and resuspended in 50 μl/well.

The cells were run on an IQue3 and tau containing cells were gated via the alexa-488 fluorescence from HT7 and tau binding assessed via alexa-647 staining. The data was normalised and expressed as a percentage of the binding signal observed for not-mutated tau.

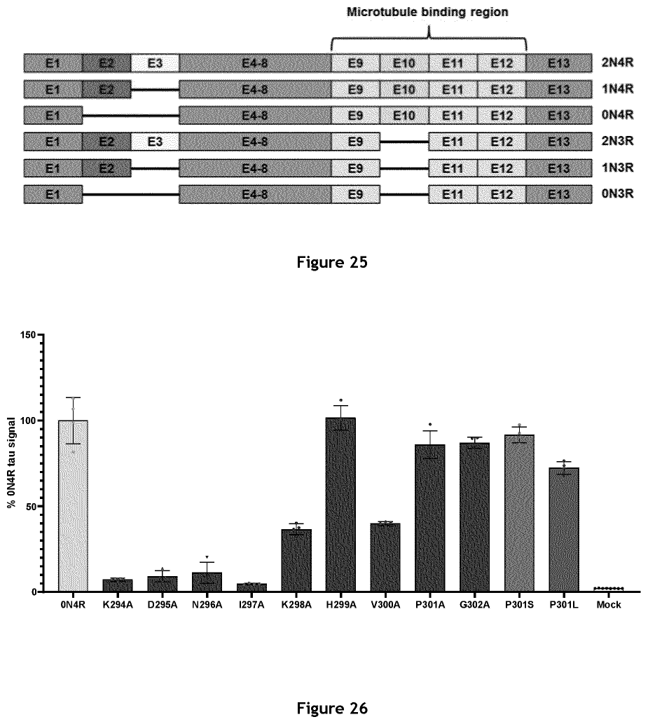

The results obtained are presented in FIG. 26. The data in FIG. 26 demonstrates the knockdown of tau binding signal in the presence of a single specific alanine mutation across the peptide-binding site (2^{nd} to 10^{th} bars going from left to right), the binding to P301S tau (11^{th} bar going from left to right), P301L tau (12^{th bar going from left to right) and mock-transfected cells (final bar going from left to right). All values are expressed as a percentage of binding to unmutated} 0N4 R tau (the 1^{st} bar going from left to right). The results show that mutation of any of K294; D295, N296, and I297 to alanine almost completely ablates binding of VR7082 antibody to tau. Mutation of either of K298 and V300 to alanine roughly halves binding of VR7082 antibody to tau. Mutation of H299, P301, and G302 did not affect binding of VR7082 to tau. The results also demonstrated that VR7082 is capable of binding both P301S and P301L indicating that it may be employed in animal or cellular models of tauopathies containing those mutations.

1. An antibody or antigen-binding fragment thereof that:
(a) specifically binds 4 R tau protein isoforms in a physiological sample; or
(b) specifically binds 3 R tau protein isoforms in a physiological sample.

2. The antibody or antigen-binding fragment thereof of claim 1, wherein the antibody or antigen-binding fragment thereof specifically binds 4 R tau protein isoforms.

3. The antibody or antigen-binding fragment thereof of claim 2, wherein the antibody or antigen-binding fragment thereof:
(a) binds to the amino acid region encoded by exon 10 of tau when the 4 R tau protein contains a post translational modification at one or more of amino acid positions 279, 280, 281, 285, and 289 of tau 4 R;
(b) binds to a peptide comprising, or consisting of, amino acids 294 to 302 of 4 R tau protein;
(c) binds to a peptide corresponding to a single epitope from the amino acid sequence encoded by exon 10 of tau; or
(d) cross-blocks or is cross-blocked by any of the antibodies or fragments of (a) to (c).

4. The antibody or antigen-binding fragment thereof of claim 2, wherein:
(a) the antibody or antigen-binding fragment specifically binds 4 R tau protein isoforms in cell lysates from cells expressing physiological 4 R tau protein isoform(s), preferably in cell lysates from iPSC derived neuronal cells expressing 4 R tau protein isoform(s); and/or
(b) the antibody or antigen-binding fragment is able to detect 4 R tau protein isoforms via immunofluorescence on, or in, cells expressing 4 R tau protein isoform(s).

5. The antibody or antigen-binding fragment thereof of claim 2, wherein the antibody or antigen binding fragment thereof:
(a) comprises one or more of the light chain CDR1, CDR2, and CDR3 sequences of SEQ ID NOs: 3, 5, and 7 and one or more of the heavy chain CDR1, CDR2, and CDR3 sequences of SEQ ID NOs: 11, 13, and 15, and preferably comprises the light chain CDR1, CDR2, and CDR3 sequences of SEQ ID NOs: 3, 5, and 7 and heavy chain CDR1, CDR2, and CDR3 sequences of SEQ ID NOs: 11, 13, and 15;
(b) comprises the light and heavy chain variable regions of SEQ ID NOs: 1 and 9;
(c) is an intrabody, preferably comprising the sequence of SEQ ID NO: 17 or a sequence with at least 95% sequence identity thereto that is still able to specifically bind tau protein 4 R isoforms; or
(d) cross-blocks or is cross-blocked by any of the antibodies or fragments of (a) to (c).

6. The antibody or antigen-binding fragment thereof of claim 2, wherein the antibody or antigen-binding fragment thereof binding an epitope of 4 R tau protein comprising amino acids K294, D295, N296, and 1297.

7. The antibody or antigen-binding fragment thereof of claim 2, wherein the epitope further comprises K298 and V300 of 4 R tau protein.

8. The antibody or antigen-binding fragment thereof of claim 1, wherein the antibody or antigen-binding fragment thereof specifically binds 3 R tau protein isoforms.

9. The antibody or antigen-binding fragment thereof of claim 8, wherein the antibody or antigen-binding fragment thereof:
(a) binds to a peptide that comprises the amino acid sequence encoded by the region bridging exons 9 and 11 of tau; and/or
(b) binds to a peptide consisting of seven amino acids either side of the boundary of the sequence encoded by exons 9 and 11 of tau.

10. The antibody or antigen-binding fragment thereof of claim 9, wherein the peptide is a cyclic peptide.

11. The antibody or antigen-binding fragment thereof of claim 8, wherein the antibody or antigen binding fragment thereof:
(a) comprises one or more of the light chain CDR1, CDR2, and CDR3 sequences of SEQ ID-NOs NOs: 20, 22, and 24 and one or more of the heavy chain CDR1, CDR2, and CDR3 sequences of SEQ ID NOs: 28, 30, and 32, and preferably comprises the light chain CDR1, CDR2, and CDR3 sequences of SEQ ID NOs: 20, 22, and 24 and heavy chain CDR1, CDR2, and CDR 3 sequences of SEQ ID NOs: 28, 30, and 32;
(b) comprises the light and heavy chain variable regions of SEQ ID NOs: 18 and 26;
(c) is an intrabody, preferably comprising the sequence of SEQ ID NO: 34 or a sequence with at least 95% sequence identity thereto that is able to specifically bind tau protein 3 R isoforms; or
(d) cross-blocks or is cross-blocked by any of the antibodies or fragments of (a) to (c).

12. An antibody or antigen-binding fragment thereof that specifically binds 4 R tau protein, wherein the antibody or antigen-binding fragment thereof binds an epitope of 4 R tau protein comprising amino acids K294, D295, N296, and 1297, optionally where the epitope further comprises K298 and V300 of 4 R tau protein.

13. An antibody or antigen-binding fragment thereof according to claim 1, wherein the antibody is an intrabody or a degrabody.

14. A nucleic acid or nucleic acids encoding an antibody or antigen binding fragment according to claim 1.

15. A vector or vectors comprising a nucleic acid or nucleic acids according to claim 14.

16. A host cell comprising a nucleic acid or nucleic acids according to claim 14.

17. A pharmaceutical composition comprising:
(a) an antibody or antigen-binding fragment thereof according to claim 1; and
(b) a pharmaceutical carrier or excipient.

18. A method of detecting 4 R tau protein isoforms comprising:
(a) contacting a test sample with an antibody or antigen-binding fragment thereof according to claim 2; and
(b) detecting binding of the antibody or antigen-binding fragment thereof.

19. A method of detecting 3 R tau protein isoforms comprising:
(a) contacting a test sample with the antibody or antigen-binding fragment thereof of claim 8; and
(b) detecting binding of the antibody or antigen-binding fragment thereof.

20. A method of determining the levels of 4 R and 3 R tau protein isoforms comprising performing the method of claim 18.

21. The method of claim 19 which further comprises determining whether the relative amount of 4 R and 3 R tau protein isoforms is different from that expected for a subject, optionally by comparing to the relative amount of 4 R and 3 R in a sample from a healthy subject.

22. The method of claim 19, wherein the method is used to diagnose tauopathies, preferably PSP, CBD, or Pick's Disease.

23. The method ofany one of claim 18 wherein:
(a) the detection is via immunofluorescence;
(b) the detection is via a Peggy Sue Simple Western blot;
(c) the detection is via ELISA; or
(c) the test sample comprises a cell lysate, cells, or tissue.

24-25. (canceled)

26. A method of treating a tauopathy comprising administering an antibody or antigen-binding fragment thereof according to claim 1 to a subject with the tauopathy.

27. The method of claim 26, wherein the tauopathy comprises an imbalance between 4 R tau protein isoforms and 3 R tau protein isoforms.

28-29. (canceled)

30. A host cell comprising a vector or vectors according to claim 15.

31. A pharmaceutical composition comprising:
(a) a nucleic acid or nucleic acids according to claim 14; and
(b) a pharmaceutical carrier or excipient.

32. A pharmaceutical composition comprising:
(a) a vector or vectors according to claim 15; and
(b) a pharmaceutical carrier or excipient.

33. A method of treating a tauopathy comprising administering a nucleic acid or nucleic acids according to claim 14 to a subject with the tauopathy.

34. The method of claim 33, wherein the tauopathy comprises an imbalance between 4 R tau protein isoforms and 3 R tau protein isoforms.

35. A method of treating a tauopathy comprising administering a vector or vectors according to claim 15 to a subject with the tauopathy.

36. The method of claim 35, wherein the tauopathy comprises an imbalance between 4 R tau protein isoforms and 3 R tau protein isoforms.

37. A method of treating a tauopathy comprising administering a pharmaceutical composition according to claim 17 to a subject with the tauopathy.

38. The method of claim 37, wherein the tauopathy comprises an imbalance between 4 R tau protein isoforms and 3 R tau protein isoforms.


SEQUENCE LISTING (59 sequences)

SEQ ID NO: 1 - protein, 109 aa, Artificial Sequence
  VR7082 - Light chain variable region
AVVLTQTPASVEAAVGGTVSINCQASEDIESYLAWYQQKPGQRPKLLIYRASTLASGVSSRFKGSGSGTEYTLTISGVECADAATYYCQEGYSIHVDNVFGGGTEVVVK

SEQ ID NO: 2 - protein, 23 aa, Artificial Sequence
  VR7082 - FW1
AVVLTQTPASVEAAVGGTVSINC

SEQ ID NO: 3 - protein, 11 aa, Artificial Sequence
  VR7082 - CDR1
QASEDIESYLA

SEQ ID NO: 4 - protein, 15 aa, Artificial Sequence
  VR7082 - FW2
WYQQKPGQRPKLLIY

SEQ ID NO: 5 - protein, 7 aa, Artificial Sequence
  VR7082 - CDR2
RASTLAS

SEQ ID NO: 6 - protein, 32 aa, Artificial Sequence
  VR7082 - FW3
GVSSRFKGSGSGTEYTLTISGVECADAATYYC

SEQ ID NO: 7 - protein, 11 aa, Artificial Sequence
  VR7082 - CDR3
QEGYSIHVDNV

SEQ ID NO: 8 - protein, 10 aa, Artificial Sequence
  VR7082 - FW4
FGGGTEVVVK

SEQ ID NO: 9 - protein, 120 aa, Artificial Sequence
  VR7082 - Heavy chain variable region
QSVEESGGRLVTPGTPLTLTCTVSGIDLSNYAMIWVRQAPGEGLEYIGIISSTGNTFYATWAKGRFTISKTSTTVDLKITSPTAEDTATYFCARDTNSDSHYGWEAFNIWGPGTLVTVSS

SEQ ID NO: 10 - protein, 24 aa, Artificial Sequence
  VR7082 - FW1
QSVEESGGRLVTPGTPLTLTCTVS

SEQ ID NO: 11 - protein, 10 aa, Artificial Sequence
  VR7082 - CDR1
GIDLSNYAMI

SEQ ID NO: 12 - protein, 14 aa, Artificial Sequence
  VR7082 - FW2
WVRQAPGEGLEYIG

SEQ ID NO: 13 - protein, 16 aa, Artificial Sequence
  VR7082 - CDR2
IISSTGNTFYATWAKG

SEQ ID NO: 14 - protein, 30 aa, Artificial Sequence
  VR7082 - FW3
RFTISKTSTTVDLKITSPTAEDTATYFCAR

SEQ ID NO: 15 - protein, 15 aa, Artificial Sequence
  VR7082 - CDR3
DTNSDSHYGWEAFNI

SEQ ID NO: 16 - protein, 11 aa, Artificial Sequence
  VR7082 - FW4
WGPGTLVTVSS

SEQ ID NO: 17 - protein, 251 aa, Artificial Sequence
  VR7082 - derived scFv intrabody
MAQSVEESGGRLVTPGTPLTLTCTVSGIDLSNYAMIWVRQAPGEGLEYIGIISSTGNTFYATWAKGRFTISKTSTTVDLKITSPTAEDTATYFCARDTNSDSHYGWEAFNIWGPGTLVTVSSGGGGSGGGGSGGGGSGGGGSAVVLTQTPASVEAAVGGTVSINCQASEDIESYLAWYQQKPGQRPKLLIYRASTLASGVSSRFKGSGSGTEYTLTISGVECADAATYYCQEGYSIHVDNVFGGGTEVVVK

SEQ ID NO: 18 - protein, 112 aa, Artificial Sequence
  VR7081 - Light chain variable region
AQVLTQTPASVSAAVGGTVSISCQSSPSVLNTNDLSWFQQKPGQPPKVLIYGASTLASGVPSRFKGSGSGTQFTLTISDVQCDDAATYYCAGGYDVNIDIYGFGGGTEVVVK

SEQ ID NO: 19 - protein, 23 aa, Artificial Sequence
  VR7081 - FW1
AQVLTQTPASVSAAVGGTVSISC

SEQ ID NO: 20 - protein, 13 aa, Artificial Sequence
  VR7081 - CDR1
QSSPSVLNTNDLS

SEQ ID NO: 21 - protein, 15 aa, Artificial Sequence
  VR7081 - FW2
WFQQKPGQPPKVLIY

SEQ ID NO: 22 - protein, 7 aa, Artificial Sequence
  VR7081 - CDR2
GASTLAS

SEQ ID NO: 23 - protein, 32 aa, Artificial Sequence
  VR7081 - FW3
GVPSRFKGSGSGTQFTLTISDVQCDDAATYYC

SEQ ID NO: 24 - protein, 12 aa, Artificial Sequence
  VR7081 - CDR3
AGGYDVNIDIYG

SEQ ID NO: 25 - protein, 10 aa, Artificial Sequence
  VR7081 - FW4
FGGGTEVVVK

SEQ ID NO: 26 - protein, 118 aa, Artificial Sequence
  VR7081 - Heavy chain variable region
QSLEESGGRLVTPGTPLTLTCTVSGFSLSNYAMIWVRQAPGGGLEWIGTMGTNGRTYYASWAKGRFTISKTSTTMDLSMTSLTAEDTATYFCARDAGITYSTYYFNIWGPGTLVTVSS

SEQ ID NO: 27 - protein, 24 aa, Artificial Sequence
  VR7081 - FW1
QSLEESGGRLVTPGTPLTLTCTVS

SEQ ID NO: 28 - protein, 10 aa, Artificial Sequence
  VR7081 - CDR1
GFSLSNYAMI

SEQ ID NO: 29 - protein, 14 aa, Artificial Sequence
  VR7081 - FW2
WVRQAPGGGLEWIG

SEQ ID NO: 30 - protein, 16 aa, Artificial Sequence
  VR7081 - CDR2
TMGTNGRTYYASWAKG

SEQ ID NO: 31 - protein, 30 aa, Artificial Sequence
  VR7081 - FW3
RFTISKTSTTMDLSMTSLTAEDTATYFCAR

SEQ ID NO: 32 - protein, 13 aa, Artificial Sequence
  VR7081 - CDR3
DAGITYSTYYFNI

SEQ ID NO: 33 - protein, 11 aa, Artificial Sequence
  VR7081 - FW4
WGPGTLVTVSS

SEQ ID NO: 34 - protein, 252 aa, Artificial Sequence
  VR7081 - derived scFv intrabody
MAQSLEESGGRLVTPGTPLTLTCTVSGFSLSNYAMIWVRQAPGGGLEWIGTMGTNGRTYYASWAKGRFTISKTSTTMDLSMTSLTAEDTATYFCARDAGITYSTYYFNIWGPGTLVTVSSGGGGSGGGGSGGGGSGGGGSAQVLTQTPASVSAAVGGTVSISCQSSPSVLNTNDLSWFQQKPGQPPKVLIYGASTLASGVPSRFKGSGSGTQFTLTISDVQCDDAATYYCAGGYDVNIDIYGFGGGTEVVVK

SEQ ID NO: 35 - protein, 441 aa, Homo Sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 36 - protein, 14 aa, Homo Sapiens
HQPGGGKVQIVYKP

SEQ ID NO: 37 - protein, 9 aa, Homo Sapiens
KDNIKHCPG

SEQ ID NO: 38 - protein, 94 aa, Homo Sapiens
LQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGG

SEQ ID NO: 39 - protein, 125 aa, Homo Sapiens
LQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGG

SEQ ID NO: 40 - protein, 57 aa, Artificial Sequence
  Clone 2
YFCARDTNSDSHYGWEAFNIWGPGTLVTVSSYYCQEGYSIHVDNVFGGGTEVVVKRT

SEQ ID NO: 41 - protein, 54 aa, Artificial Sequence
  Clone 10
YFCARAADTATSGYDIWRPGTLDTVSSYYCQCTDYGSTYIGAFGGGTEVVVART

SEQ ID NO: 42 - protein, 57 aa, Artificial Sequence
  Clone 8
YFCARVRGNSRYYFMDLWGQGTLVTVSSYYCESNYWSFNTNYGAPFGGGTEVVVKRT

SEQ ID NO: 43 - protein, 55 aa, Artificial Sequence
  Clone 5
YFCARWTTKYAEDGYDIWSPGTLVTVSSYYCAGCKSTTIDSNTFGGGTEVVVKRT

SEQ ID NO: 44 - protein, 57 aa, Artificial Sequence
  Clone 4
YFCARWTTKYAEDGYDIWRPGTLVTVSSYYCQSNYWSFNTNYGAPFGGGTEVVVKRT

SEQ ID NO: 45 - protein, 57 aa, Artificial Sequence
  Clone 9
YFCARWTTKYAEDGYDIWSPGTLVTVSSYYCQSNYWSFNTNYGAPFGGGTEVVVKRT

SEQ ID NO: 46 - protein, 57 aa, Artificial Sequence
  Clone 1
YFCARGTTKYAEDGYDIWGPGTLVTVSSYYCQSNYWSFNTNYGAPFGGGTEVVVRRT

SEQ ID NO: 47 - protein, 57 aa, Artificial Sequence
  Clone 7
YFCARGTTKYAEDGYDIWGPGTLVTVSSYYCQSNYWSFNTNYGAPFGGGTEVVVKRT

SEQ ID NO: 48 - protein, 57 aa, Artificial Sequence
  Clone 6
YFCARGTTKYAEDGYDIWGPGTLVTVSSYYCQSNYGSFNTNYGAPFGGGTEVVVKRT

SEQ ID NO: 49 - protein, 56 aa, Artificial Sequence
  Clone 11
YFCARDAGITYSTYYFNIQGPGTLVTVSSYYCAGDYDVNIDIYGFGGGTEVVVKRT

SEQ ID NO: 50 - protein, 45 aa, Artificial Sequence
  Clone 13
YFCARGLEMWGPGTLVTVSSYYCVGDFSGWIFAFGGGTEVVVKRT

SEQ ID NO: 51 - protein, 45 aa, Artificial Sequence
  Clone 12
YFCARGLEMWGPGTLVTVSSYYCLGGYYGHIYVFGGGTEVVVKRT

SEQ ID NO: 52 - protein, 45 aa, Artificial Sequence
  Clone 15
YFCARGLEIWGPGTLVTVSSYYCLGGYIDHIYVFGGGTEVVVVRT

SEQ ID NO: 53 - protein, 45 aa, Artificial Sequence
  Clone 18
YFCARGLEIWGPGTLVTVSSYYCLGGYSGHIYVFGGGTEVVVKRT

SEQ ID NO: 54 - protein, 45 aa, Artificial Sequence
  Clone 19
YFCARDLEIWGPGTLVTVSSYYCLGGYSGHIYVFGGGTEVVVKRT

SEQ ID NO: 55 - protein, 45 aa, Artificial Sequence
  Clone 17
YFCGRGLEIWGPGTLVTVSSYYCLGGYSGHIYVFGGGTEVVVKRT

SEQ ID NO: 56 - protein, 45 aa, Artificial Sequence
  Clone 16
YFCGRGLEIWGPGTLVTVSSYYCLGGYLGHVYVFGGGTEVVVKRT

SEQ ID NO: 57 - protein, 4 aa, Homo sapiens
KDEL

SEQ ID NO: 58 - protein, 6 aa, Homo sapiens
SEKDEL

SEQ ID NO: 59 - protein, 5 aa, Artificial Sequence
  Peptide Linker
GGGGS